(19) United States
(12) Patent Application Publication
(10) Pub. No.: US 2018/0371404 A1
(43) Pub. Date: Dec. 27, 2018

(54) METABOLIC ENGINEERING OF MEMBRANE PROTEINS TO IMPROVE ESCHERICHIA COLI MEMBRANE INTEGRITY AND PRODUCTION OF FATTY ACIDS

(71) Applicant: Iowa State University Research Foundation, Inc., Ames, IA (US)

(72) Inventors: Laura Jarboe, Ames, IA (US); Zaigao Tan, Ames, IA (US); Jacqueline V. Shanks, Ames, IA (US)

(21) Appl. No.: 16/018,540

(22) Filed: Jun. 26, 2018

Related U.S. Application Data
(60) Provisional application No. 62/525,486, filed on Jun. 27, 2017.

(51) Int. Cl. C12N 1/20; C12N 15/10; C12N 15/74; C12N 15/70; C12R 1/19; C12R 1/07; C12R 1/145

(52) U.S. Cl. CPC C12N 1/20; C12N 15/102; C12N 15/746; C12N 2310/20; C12R 1/19; C12R 1/07; C12R 1/145; C12N 15/70

(57) ABSTRACT

The present disclosure provides bacteria with improved membrane integrity related primarily to outer membrane protein content and increased fatty acid production. According to the invention, Applicants have successfully demonstrated that modulation of membrane protein FADL increased membrane integrity and final fatty acid titre by 34%, Surprisingly, applicants also showed that combining the FADL regulation with a decrease or inactivation of OmpF in bacterial membranes resulted in a synergistic increase of 53% fatty acid production. The invention also provides for an isolated or recombinant bacterium, and use of the same for fatty acid production.

### CROSS-REFERENCE TO RELATED APPLICATIONS

This application claims priority under 35 U.S.C. § 119 to provisional application U.S. Ser. No. 62/525,486, filed Jun. 27, 2017, herein incorporated by reference in its entirety.

### GRANT REFERENCE

This invention was made with government support under contract NSF Grant No. EECO813570. The Government has certain rights in this invention.

### FIELD OF THE INVENTION

The present disclosure relates to genetically engineered bacteria for biorenewable production of organic chemicals.

### BACKGROUND OF THE INVENTION

Long term economic and environmental concerns with the current petroleum-based economy have driven the development of approaches that convert renewable sources to organic chemicals to replace those derived from petroleum feed stocks. Production of biofuels, such as ethanol or butanol, by microorganisms has been a research focus in recent years, and significant progress has been made in this area. At the same time, there remains a great need for development of biorefining processes that utilize microorganisms to convert renewable sources into industrially useful chemicals.

Esters, in particular, are in great demand for a variety of industrial and cosmetic applications. Isobutyl isobutyrate is a popular retarder solvent for a variety of lacquers and thinners, and it can be found in numerous painted coatings. It is also used as an insect repellant. The current method of producing these esters involves individually producing the constituent alcohols and carboxylic acids, then esterifying them through chemical synthesis or enzymatic reactions. This two-step process requires individual production and purification steps to generate each constituent and further processing and purification steps to yield the final ester. Much of the production of the constituent alcohols and carboxylic acids depends heavily upon petroleum, which is increasingly expensive and associated with global health and environmental concerns.

Although many successes have been achieved during the past few decades, strain performance has been still restricted by some limiting factors, e.g. inhibitors in feedstock and toxicity from bio-products. Furfural, which is an inhibitory by-product formed by dehydration of xylose during treatment of lignocellulose by dilute acid, inhibits E. coli LY01 growth completely at the concentration of 3.5 g/L (Wang 2011 Zaldivar 1999). The specific growth rate of E. coli Frag1 was reduced by 50% when challenged with only 8 mM acetate (Roe 2002). For bio-products, in the presence of 35 g/L ethanol, engineered E. coli KO11 grows poorly and less than 10% of KO11 cells survived challenge with 100 g/L of ethanol (10). In our previous study, we also found that at the concentration of 40 mM, short-chain hexanoic acid (C6) and octanoic acid (C8) are completely inhibitory to E. coli MG1655 (Royce 2013).

A diversity of detrimental effects are caused by toxic solvents to host strains, e.g. DNA damage, RNA degradation, membrane damage, energy loss. Solvents can intercalate in the phospholipid bilayer of membrane thus disrupting the membrane electrochemical potential (Δψ), proton potential (ΔpH), and inhibiting membrane functions (Nicolaou 2010). Ethanol was considered to fluidize the cell membrane, leading to arbitrary transport of solutes, which decreased the membrane potential, proton gradient across the membrane and caused outflow of important ions such as Mg2+(Huffer 2011). Brynildsen et al. utilized transcriptional analysis to investigate the mechanism of isobutanol toxicity and deemed that membrane damage is an important reason for isobutanol toxicity, which might be due to disruption of the electron transport chain (Brynildsen 2009; Jarboe 2011). Lennen et al. reported that the membrane integrity markedly decreased during fatty acids production and cell viability decreased by 85% compared with control strain.

Fatty acids, which serve as catalytic precursors for a variety of chemicals, are widely used in production of lubricants, preservatives, fuels. However, as stated above, this chemical was also reported to cause membrane damage of E. coli during production. Proceeding engineering researches have attempted to engineer membrane fatty acid tails of phospholipids, for the purpose of alleviating toxicity, with mixed results. Lennen et al. reported that expression of a second thioesterase (GeoTE), which impedes medium chain length, unsaturated acyl-ACPs from being incorporated into the membrane, restored normal membrane content and thus increased cellular integrity in the course of fatty acid production. Although this strategy was effective in alleviating membrane leakage, no significant increase in fatty acids production was observed. Sherkhanov et al. found that deletion of aas, which is responsible for incorporating fatty acids with medium chain length into membrane improved the average lipids length of membrane, alleviated the toxicity of fatty acids and increased fatty acids titer by 20%.

As can be seen there is a continuing need to develop and engineer bacteria for improved production of valuable biorenewables.

### BRIEF SUMMARY OF THE INVENTION

The present disclosure provides bacteria with improved membrane integrity related primarily to outer membrane protein content and concomitant increased fatty acid production. These modified strains exhibit up to 50% increase in fatty acid production. Methods for the production of the bacteria, as well as uses thereof for production of biorenewables are also provided.

According to the invention, Applicants have successfully demonstrated that modulation of two distinct outer membrane proteins, membrane transporter protein FADL (long chain fatty acid outer membrane transporter) and OmpF (outer membrane porin F) in a precise manner can increase membrane integrity and final fatty acid titre in a synergistic manner. The invention also provides for an isolated or recombinant bacterium, wherein the bacterium has increased activity/abundance of FADL and decreased activity/abundance of OmpF as compared to a reference bacterium.

In another embodiment, the modulating comprises: (a) introducing into the bacterium an expression construct comprising at least one FADL encoding polynucleotide sequence, or a subsequence thereof, operably linked to a heterologous promoter, which promoter functions in bacteria and/or, (b) expressing at least one polynucleotide sequence, thereby modulating (increasing) the activity of one or more FADL encoding polynucleotides to increase FADL production compared to a corresponding control bacterium (e.g., its non-modified parent or a non-modified bacterium of the same species). For example, at least one polynucleotide sequence can be introduced by standard techniques including, but not limited to, electroporation, micro-projectile bombardment, bacteriophage-mediated transfer, and the like.

With respect to the OmpF the modulating comprises reducing or inactivating the protein. The protein can be produced by effecting deletion, insertion, inversion, substitution, derivatization, and/or or any other change of amino acids at the amino acid level in order to deactivate the transporter protein. Alternatively, inactivation can occur at the nucleic acid level by genetic modification (also referred to herein as mutation). Genetic modification includes deletion, insertion, inversion, substitution, derivatization, and/or any other change in a gene encoding the transporter protein such that the genetically modified nucleic acid does not encode an active transporter protein. In some embodiments, no transporter protein is produced at all. In some embodiments, the encoded transporter protein is truncated.

The invention provides for an isolated or recombinant bacterium, wherein the activity of FADL proteins is increased and the activity of OmpF protein is reduced as compared to a reference bacterium. The bacterium has increased short chain fatty acid production and viability. According to the invention, the OmpF deletion increases cell viability in an environment with moderate levels of short chain fatty acids, this combined with overexpression of FADL increases the import of long chain fatty acids to bolster the cell membrane and synergistically boost fatty acid production.

The invention also provides for an isolated or recombinant bacterium wherein the production of biorenewables is increased as compared to the reference bacterium, the biorenewables are produced by a process comprising the steps of:

(a) growing a modified bacteria strain of the invention; and
(b) harvesting biorenewables produced by said strain.

### DESCRIPTION OF THE DRAWINGS

FIG. 1: Effects of ompF or fadL deletion on membrane integrity during short-chain fatty acid challenge, short-chain fatty acid tolerance and production of C12 and C14 fatty acids.

(A) Deletion of ompF or fadL impact the specific growth rate relative to the wild type MG1655 during challenge with 10 mM C8. Inset values are the specific growth rate, h-1
(B) Deletion of ompF or fadL alters the percentage of cells with intact membranes (membrane integrity), assessed using SYTOX Green, during challenge with 10 mM C8.
(C) Deletion of ompF increased fatty acid production and deletion of fadL decreased fatty acid production. MG1655+TE-1 and MG1655+TE-2 indicates experiments performed with the same strain, but on different days. For (A) and (B), experiments were performed in MOPS+2% (wt/v) dextrose shake flasks at 220 rpm 30° C. with an initial pH of 7.0, 10 mM octanoic acid (C8). For (C), strains carry the pXZ18Z plasmid (TE) for LCFA (C14-C16) production. Fermentations were performed in MOPS+2% (wt/v) dextrose shake flasks at 220 rpm 30° C. with an initial pH of 7.0, 1.0 mM IPTG. Values are the average of at least three biological replicates with error bars indicating one standard deviation. Percent increase values are shown only for differences that were deemed statistically significant (P<0.05).

FIG. 2: Increased expression of fadL increases membrane integrity, fatty acid tolerance and production. (A) Increased expression of fadL from a plasmid (Pla-fadL) or a genomic insertion (Gen-fadL) both increase the specific growth rate relative to the corresponding controls (Pla-empty, Gen-empty) during challenge with 10 mM C8. Inset values are the specific growth rate, h-1. (B) Percentage of cells with intact membrane (membrane integrity), assessed using SYTOX Green. Strains with increased expression of fadL, Pla-fadL and Gen-fadL, have improved membrane integrity relative to their corresponding controls, Pla-empty and Gen-empty, during challenge with 10 mM C8. (C) Strains with increased expression of fadL, Pla-fadL and Gen-fadL, produce more fatty acid than the corresponding controls, Pla-empty and Gen-empty. (D) The fadL mRNA relative abundance at 48 h has a positive relationship with the fatty acids titer after 72 h. Four different promoters (M1-12, M1-37, M1-46 and M1-93) were used to replace the native promoter of fadL. The mRNA abundance of fadL in M1-12-fadL strain was set as 1. The 16S rrsA gene was used as normalizing factor. For (A) and (B), experiments were performed in shake flasks containing MOPS+2% (wt/v) dextrose with 10 mM octanoic acid (C8) at an initial pH of 7.0, shaken at 220 rpm, and maintained at 30° C. For (C) and (D), all strains carry the pXZ18Z plasmid (TE, fabZ) for LCFA (C14-C16) production. Fermentations were performed in MOPS+2% (wt/v) dextrose shake flasks at 220 rpm 30° C. with an initial pH of 7.0, 1.0 mM IPTG. Values are the average of at least three biological replicates with error bars indicating one standard deviation. Percent increase values are shown only for differences that were deemed statistically significant (P<0.05). Pla-empty: MG1655+pACYC184-Kan; Pla-fadL: MG1655+pACYC184-Kan-fadL; Gen-empty: MG1655 ldhA: FRT-cat-FRT; Gen-fadL: MG1655 ldhA: FRT-cat-FRT, fadL. TE: pXZ18Z plasmid.

FIG. 3: Deletion of ompF and increased expression of fadL have an additive effect on increasing membrane integrity, fatty acid tolerance and production.

(A) Combinatorial deletion of ompF (ΔompF) and increased expression of fadL (Pla-fadL) increases the specific growth rate during challenge with 10 mM C8 relative to the starting strain (Pla-empty), individual ompF deletion strain (ΔompF+Pla-empty), and individual overexpression of fadL (Pla-fadL). Inset values are the specific growth rate, h-1 (B) Percentage of cells with intact membrane (membrane integrity), assessed using SYTOX Green. Combinatorial deletion of ompF and increased expression of fadL improves membrane integrity during challenge with 10 mM C8 relative to Pla-empty, ΔompF+Pla-empty and Pla-fadL strains. (C) The combined implementation of ompF deletion and increased expression of fadL supports increased fatty acid titers relative to each engineering strategy implemented individually. For (A) and (B), experiments were performed in MOPS+2% (wt/v) dextrose shake flasks at 220 rpm 30° C. with an initial pH of 7.0, 10 mM octanoic acid (C8). For (C), all strains carry the pXZ18Z plasmid (TE, fabZ) for LCFA (C14-C16) production. Fermentations were performed in MOPS+2% (wt/v) dextrose shake flasks at 220 rpm 30° C. with an initial pH of 7.0, 1.0 mM IPTG. Values are the average of at least three biological replicates with error bars indicating one standard deviation. Percent increase values are shown only for differences that were deemed statistically significant (P<0.05). Pla-empty: MG1655+pACYC184-Kan; ΔompF+Pla-empty: MG1655, ΔompF+pACYC184-Kan; Pla-fadL: MG1655+pACYC184-Kan-fadL; ΔompF+Pla-fadL: MG1655, ΔompF+pACYC184-Kan-fadL.

FIG. 4: Schematic of the proposed role of ompF and fadL in maintenance of membrane integrity during fatty acid production in E. coli.

The elongated acyl-ACP formed during the fatty acids biosynthesis will have two major destinations. Partial acyl-ACPs are hydrolyzed by thioesterase to release free fatty acids. Residual acyl-ACPs serve as precursor for membrane lipids biosynthesis. Among the produced free fatty acids, LCFA (C14-C16) predominates while there is still some SCFA (<C10). It is proposed that LCFA and SCFA are both transported from the cytoplasm directly to the extracellular medium with the AcrAB-TolC complex [26]. However, the low abundance of these compounds in the periplasmic space relative to the extracellular medium results in a driving force for SCFA entry via OmpF and LCFA entry via FadL. LCFAs imported by FadL can be catalyzed by FadD to acyl-CoA, which then serve as fatty acyl precursors for synthesis of phospholipids or enter the β-oxidation cycle for degradation. SCFAs that enter the cell through OmpF, can damage the inner membrane. Increased expression of fadL contributes to import of exogenous LCFA, providing precursors for membrane lipids biosynthesis, thereby increasing membrane integrity and supporting fatty acids production, while deletion of OmpF prevents re-entry of the harmful SCFA. LCFA, long chain fatty acids; SCFA, short-chain fatty acids.

FIG. 5 Fatty acids profile of E. coli MG1655 harboring pXZ18Z plasmid which carries thioesterase gene from Ricinus communis and fabZ gene from E. coli. Some short chain fatty acids (e.g. butanedioic acid, octanoic acid and decanoic acid) were found in the fermentation broth.

### DETAILED DESCRIPTION OF THE INVENTION

### I. Definitions

As used herein, “isolated” means free from contamination by other bacteria. An isolated bacterium can exist in the presence of a small fraction of other bacteria which do not interfere with the properties and function of the isolated bacterium. An isolated bacterium will generally be at least 30%, 40%, 50%, 60%, 70%, 80%, 85%, 90%, 95%, 98%, or 99% pure. Preferably, an isolated bacterium according to the invention will be at least 98% or at least 99% pure.

As used herein, “bacterium” includes “non-recombinant bacterium”, “recombinant bacterium” and “modified bacterium”.

As used herein, “non-recombinant bacterium” includes a bacterial cell that does not contain heterologous polynucleotide sequences, and is suitable for further modification using the compositions and methods of the invention, e.g. suitable for genetic manipulation, e.g., which can incorporate heterologous polynucleotide sequences, e.g., which can be transfected.

As used herein, “recombinant” as it refers to bacterium, means a bacterial cell that is suitable for, or subjected to, genetic manipulation, or incorporates a heterologous polynucleotide sequence, or that has been treated such that a native polynucleotide sequence has been mutated or deleted. The term is intended to include progeny of the cell originally transfected. In particular embodiments, the cell is a Gram-negative bacterial cell or a Gram-positive bacterial cell.

As used herein, “modified” as it refers to bacterium, means a bacterial cell that is not identical to a reference bacterium, as defined herein below.

A “modified” bacterium includes a “recombinant” bacterium.

As used herein, “modified nucleic acid molecule” or “modified gene” is intended to include a nucleic acid molecule or gene having a nucleotide sequence which includes at least one alteration (e.g., substitution, insertion, deletion) such that the polypeptide or polypeptide that can be encoded by the modified exhibits an activity or property that differs from the polypeptide or polypeptide encoded by the wild-type nucleic acid molecule or gene.

As used herein, “mutation” as it refers to a nucleic acid molecule or gene means alteration, insertion or deletion of a nucleic acid or a gene, or an increase or decrease in the level of expression of a nucleic acid or a gene, wherein the increase or decrease in expression results in a respective increase or decrease in the expression of the polypeptide that can be encoded by the nucleic acid molecule or gene. A mutation also means a nucleic acid molecule or gene having a nucleotide sequence which includes at least one alteration (e.g., substitution, insertion, deletion) such that the polypeptide or polypeptide that can be encoded by the modified exhibits an activity or property that differs from the polypeptide or polypeptide encoded by the wild-type nucleic acid molecule or gene.

As used herein, “modified protein” or “modified protein or amino acid sequence” is intended to include an amino acid sequence which includes at least one alteration (e.g., substitution, insertion, deletion) such that the polypeptide or polypeptide that can be encoded by the modified amino acid sequence exhibits an activity or property that differs from the polypeptide or polypeptide encoded by the wild-type amino acid sequence.

As used herein, “mutation” as it refers to a protein or amino acid sequence means alteration, insertion or deletion of an amino acid of an amino acid sequence, or an increase or decrease in the level of expression of an amino acid sequence, wherein the increase or decrease in expression results in an increase or decrease in the expression of the polypeptide that can be encoded by amino acid sequence. A mutation also means a protein or amino acid sequence having an amino acid sequence which includes at least one alteration (e.g., substitution, insertion, deletion) such that the polypeptide or polypeptide that can be encoded by the modified exhibits an activity or property that differs from the polypeptide or polypeptide encoded by the wild-type amino acid sequence.

As used herein, “fragment” or “subsequence” is intended to include a portion of parental or reference nucleic acid sequence or amino acid sequence, or a portion of polypeptide or gene, which encodes or retains a biological function or property of the parental or reference sequence, polypeptide or gene.

A “modified” bacterium includes a bacterium of its ancestors which comprise a “mutation” as defined hereinabove.

As used herein, “reference” or “reference bacterium” includes, at least, a wild-type bacterium and a parental bacterium.

As used herein, “wild-type” means the typical form of an organism or strain, for example a bacterium, gene, or characteristic as it occurs in nature, in the absence of mutations. “Wild type” refers to the most common phenotype in the natural population. Wild type is the standard of reference for the genotype and phenotype.

As used herein, “parental” or “parental bacterium” refers to the bacterium that gives rise to a bacterium of interest.

A “gene,” as used herein, is a nucleic acid that can direct synthesis of a transporter protein or other polypeptide molecule, e.g., can comprise coding sequences, for example, a contiguous open reading frame (ORF) that encodes a polypeptide, a subsequence thereof, or can itself be functional in the organism. A gene in an organism can be clustered in an operon, as defined herein, wherein the operon is separated from other genes and/or operons by intergenic DNA. Individual genes contained within an operon can overlap without intergenic DNA between the individual genes. In addition, the term “gene” is intended to include a specific gene for a selected purpose. A gene can be endogenous to the host cell or can be recombinantly introduced into the host cell, e.g., as a plasmid maintained episomally or a plasmid (or fragment thereof) that is stably integrated into the genome. A heterologous gene is a gene that is introduced into a cell and is not native to the cell.

The term “nucleic acid” is intended to include nucleic acid molecules, e.g., polynucleotides which include an open reading frame encoding a polypeptide, a subsequence thereof, and can further include non-coding regulatory sequences, and introns. In addition, the terms are intended to include one or more genes that map to a functional locus. In addition, the terms are intended to include a specific gene for a selected purpose.

As used herein, “increasing” or “increases” or “increased” refers to increasing by at least 5%, for example, 5, 6, 7, 8, 9, 10, 15, 20, 25, 30, 35, 40, 45, 50, 55, 60, 65, 70, 75, 80, 85, 90, 95, 99, 100% or more, for example, as compared to the level of expression of the FADL genes, in a bacterium having an increased expression of the FADL genes, as compared to a reference bacterium.

As used herein, “increasing” or “increases” or “increased” also means increases by at least 1-fold, for example, 1, 2, 3, 4, 5, 6, 7, 8, 9, 10, 15, 20, 30, 40, 50, 60, 70, 80, 90, 100, 200, 500, 1000-fold or more, for example, as compared to the level of expression of the FADL genes in a bacterium, having an increased expression of a FADL gene, as compared to a reference bacterium.

As used herein, “decreasing” or “decreases” or “decreased” refers to decreasing by at least 5%, for example, 5, 6, 7, 8, 9, 10, 15, 20, 25, 30, 35, 40, 45, 50, 55, 60, 65, 70, 75, 80, 85, 90, 95, 99 or 100%, for example, as compared to the decreased level of expression of a OmpF gene in a bacterium, as compared to a reference bacterium.

As used herein, “decreasing” or “decreases” or “decreased” also means decreases by at least 1-fold, for example, 1, 2, 3, 4, 5, 6, 7, 8, 9, 10, 15, 20, 30, 40, 50, 60, 70, 80, 90, 100, 200, 500, 1000-fold or more, for example, as compared to the level of expression of a OmpF gene in a bacterium, as compared to a reference bacterium.

“Decreased” or “reduced” also means eliminated such that there is no detectable level of activity, expression, etc., for example no detectable level of expression of a OmpF gene or no detectable activity of the OmpF proteins.

As used herein, “activity” refers to the activity of a gene, for example the level of transcription of a gene. “Activity” also refers to the activity of an mRNA, for example, the level of translation of an mRNA. “Activity” also refers to the activity of a protein, for example FADL of OmpF

An “increase in activity” includes an increase in the rate and/or the level of activity.

As used herein, “expression” as in “expression of FADL of OmpF” refers to the expression of the protein product of a FADL or OmpF gene. As used herein, “expression” as in “expression of FADL of OmpF” also refers to the expression of detectable levels of the mRNA transcript corresponding to a FADL or OmpF gene.

“Altering”, as it refers to expression levels, means decreasing expression of a gene, mRNA or protein of interest, for example a FADL or OmpF gene.

As used herein, “not expressed” means there are no detectable levels of the product of a gene or mRNA of interest, for example, FADL of OmpF genes.

As used herein “eliminate” means decrease to a level that is undetectable.

As used herein, “derived from” means originates from.

The term “Gram-negative bacterial cell” is intended to include the art-recognized definition of this term. Exemplary Gram-negative bacteria include Acinetobacter, Gluconobacter, Zymomonas, Escherichia, Geobacter, Shewanella, Salmonella, Enterobacter and Klebsiella.

The term “Gram-positive bacteria” is intended to include the art-recognized definition of this term. Exemplary Gram-positive bacteria include Bacillus, Clostridium, Corynebacterium, Lactobacillus, Lactococcus, Oenococcus, Streptococcus and Eubacterium.

The term “FADL” or “FADL protein” refers to a gram-positive bacteria outer membrane protein having long chain (C14 orC16) transporter activity, as exemplified in strain MG1655 including variants, homologs, and truncations with said activity.

The term “OmpF” or “OmpF protein” refers to a gram-positive bacteria outer membrane transporter protein that transports sugars, ions, antibodies, and short chain fatty acids as exemplified in Strain MG1655 including variants, homologs, and truncations that retain said transporter activity.

The term “amino acid” is intended to include the 20 alpha-amino acids that regularly occur in proteins. Basic charged amino acids include arginine, asparagine, glutamine, histidine and lysine. Neutral charged amino acids include alanine, cysteine, glycine, isoleucine, leucine, methionine, phenylalanine, proline, serine, threonine, tryptophan, tyrosine, and valine. Acidic amino acids include aspartic acid and glutamic acid.

As used herein, “selecting” refers to the process of determining that an identified bacterium produces ethanol in the presence of furfural.

As used herein, “identifying” refers to the process of assessing a bacterium and determining that the bacterium produces ethanol in the presence of furfural.

In this disclosure, “comprises,” “comprising,” “containing” and “having” and the like have the open-ended meaning ascribed to them in U.S. patent law and mean “includes,” “including,” and the like.

For various embodiments of the invention the modified bacteria of the invention may be described to include various genetic manipulations, including those directed to change regulation of, and therefore ultimate activity/abundance of, a protein. Such genetic modifications may be directed to transcriptional, translational, and post-translational modifications that result in a change of activity and/or selectivity under selected and/or identified culture conditions and/or to provision of additional nucleic acid sequences such as to increase copy number and/or mutants of a transporter protein related to fatty acid or fatty acid derived product production. Specific methodologies and approaches to achieve such genetic modification are well known to one skilled in the art, and include, but are not limited to: increasing expression of an endogenous genetic element; decreasing functionality of a repressor gene; introducing a heterologous genetic element; increasing copy number of a nucleic acid sequence encoding a polypeptide catalyzing an enzymatic conversion step to produce fatty acid or a fatty acid derived product; mutating a genetic element to provide a mutated protein to increase specific enzymatic activity; over-expressing; under-expressing; over-expressing a chaperone; knocking out a transporter protein; altering or modifying feedback inhibition; providing an transporter protein variant comprising one or more of an impaired binding site for a repressor and/or competitive inhibitor; knocking out a repressor gene; evolution, selection and/or other approaches to improve mRNA stability as well as use of plasmids having an effective copy number and promoters to achieve an effective level of improvement. Random mutagenesis may be practiced to provide genetic modifications that may fall into any of these or other stated approaches. The genetic modifications further broadly fall into additions (including insertions), deletions (such as by a mutation) and substitutions of one or more nucleic acids in a nucleic acid of interest.

### II. Bacteria

Accordingly, the invention provides bacteria that have increased FADL membrane presence/activity by modulation of at least one FADL gene compared to a reference bacterium combined with decreased OmpF membrane presence/activity by modulation of at least one OmpF gene compared to a reference bacterium. In another aspect of the invention, the bacteria are recombinant. bacteria.

The bacteria of the invention may be characterized by their increased membrane integrity and increased production of short chain fatty acid production. The bacteria of the invention may be non-recombinant or recombinant. The bacterium of the invention are selected from the group consisting of Gram-negative bacteria and Gram-positive bacteria, wherein the Gram-negative bacterium is selected from the group consisting of Acinetobacter, Gluconobacter, Zymomonas, Escherichia, Geobacter, Shewanella, Salmonella, Enterobacter and Klebsiella and the Gram-positive bacterium is selected from the group consisting of Bacillus, Clostridium, Corynebacterium, Lactobacillus, Lactococcus, Oenococcus, Streptococcus and Eubacterium. In one aspect, the bacterium of the invention is Escherichia coli.

### III. Nucleic Acids

The present invention provides, inter alia, isolated nucleic acids of RNA, DNA, homologs, paralogs and orthologs and/or chimeras thereof, comprising a FADL or OmpF polynucleotide. This includes naturally occurring as well as synthetic variants and homologs of the sequences.

Sequences homologous, i.e., that share significant sequence identity or similarity, to those provided herein derived from bacteria or other organisms, are also an aspect of the invention. Homologous sequences can be derived from any organism including other microbes and the like.

The present invention also includes polynucleotides optimized for expression in different organisms. For example, for expression of the polynucleotide in a particular organism, the sequence can be altered to account for specific codon.

### Construction of Nucleic Acids

The isolated nucleic acids of the present invention can be made using (a) standard recombinant methods, (b) synthetic techniques, or combinations thereof. In some embodiments, the polynucleotides of the present invention will be cloned, amplified, or otherwise constructed from a fungus or bacteria.

The nucleic acids may conveniently comprise sequences in addition to a polynucleotide of the present invention. For example, a multi-cloning site comprising one or more endonuclease restriction sites may be inserted into the nucleic acid to aid in isolation of the polynucleotide. Also, translatable sequences may be inserted to aid in the isolation of the translated polynucleotide of the present invention. For example, a hexa-histidine marker sequence provides a convenient means to purify the proteins of the present invention. The nucleic acid of the present invention—excluding the polynucleotide sequence—is optionally a vector, adapter, or linker for cloning and/or expression of a polynucleotide of the present invention. Additional sequences may be added to such cloning and/or expression sequences to optimize their function in cloning and/or expression, to aid in isolation of the polynucleotide, or to improve the introduction of the polynucleotide into a cell. Typically, the length of a nucleic acid of the present invention less the length of its polynucleotide of the present invention is less than 20 kilobase pairs, often less than 15 kb, and frequently less than 10 kb. Use of cloning vectors, expression vectors, adapters, and linkers is well known in the art. Exemplary nucleic acids include such vectors as: M13, lambda ZAP Express, lambda ZAP II, lambda gt10, lambda gt11, pBK-CMV, pBK-RSV, pBluescript II, lambda DASH II, lambda EMBL 3, lambda EMBL 4, pWE15, SuperCos 1, SurfZap, Uni-ZAP, pBC, pBS+/−, pSG5, pBK, pCR-Script, pET, pSPUTK, p3′SS, pGEM, pSK+/−, pGEX, pSPORTI and II, pOPRSVI CAT, pOPI3 CAT, pXT1, pSG5, pPbac, pMbac, pMClneo, pOG44, pOG45, pFRTβGAL, pNEOβGAL, pRS403, pRS404, pRS405, pRS406, pRS413, pRS414, pRS415, pRS416, lambda MOSSlox, and lambda MOSElox. Optional vectors for the present invention, include but are not limited to, lambda ZAP II, and pGEX. For a description of various nucleic acids see, e.g., Stratagene Cloning Systems, Catalogs 1995, 1996, 1997 (La Jolla, Calif.); and, Amersham Life Sciences, Inc, Catalog '97 (Arlington Heights, Ill.).

### Synthetic Methods for Constructing Nucleic Acids

The isolated nucleic acids of the present invention can also be prepared by direct chemical synthesis by methods such as the phosphotriester method of Narang, et al., (1979) Meth. Enzymol. 68:90-9; the phosphodiester method of Brown, et al., (1979)Meth. Enzymol. 68:109-51; the diethylphosphoramidite method of Beaucage, et al., (1981) Tetra. Letts. 22(20):1859-62; the solid phase phosphoramidite triester method described by Beaucage, et al., supra, e.g., using an automated synthesizer, e.g., as described in Needham-VanDevanter, et al., (1984) Nucleic Acids Res. 12:6159-68; and, the solid support method of U.S. Pat. No. 4,458,066. Chemical synthesis generally produces a single stranded oligonucleotide. This may be converted into double stranded DNA by hybridization with a complementary sequence or by polymerization with a DNA polymerase using the single strand as a template. One of skill will recognize that while chemical synthesis of DNA is limited to sequences of about 100 bases, longer sequences may be obtained by the ligation of shorter sequences.

### UTRs and Codon Preference

In general, translational efficiency has been found to be regulated by specific sequence elements in the 5′ non-coding or untranslated region (5′ UTR) of the RNA. Positive sequence motifs include translational initiation consensus sequences (Kozak, (1987) Nucleic Acids Res. 15:8125) and the 5<G>7 methyl GpppG RNA cap structure (Drummond, et al., (1985) Nucleic Acids Res. 13:7375). Negative elements include stable intramolecular 5′ UTR stem-loop structures (Muesing, et al., (1987) Cell 48:691) and AUG sequences or short open reading frames preceded by an appropriate AUG in the 5′ UTR (Kozak, supra, Rao, et al., (1988) Mol. and Cell. Biol. 8:284). Accordingly, the present invention provides 5′ and/or 3′ UTR regions for modulation of translation of heterologous coding sequences.

Further, the polypeptide-encoding segments of the polynucleotides of the present invention can be modified to alter codon usage. Altered codon usage can be employed to alter translational efficiency and/or to optimize the coding sequence for expression in a desired host or to optimize the codon usage in a heterologous sequence for expression in rice. Codon usage in the coding regions of the polynucleotides of the present invention can be analyzed statistically using commercially available software packages such as “Codon Preference” available from the University of Wisconsin Genetics Computer Group. See, Devereaux, et al., (1984) Nucleic Acids Res. 12:387-395); or MacVector 4.1 (Eastman Kodak Co., New Haven, Conn.). Thus, the present invention provides a codon usage frequency characteristic of the coding region of at least one of the polynucleotides of the present invention. The number of polynucleotides (3 nucleotides per amino acid) that can be used to determine a codon usage frequency can be any integer from 3 to the number of polynucleotides of the present invention as provided herein. Optionally, the polynucleotides will be full-length sequences. An exemplary number of sequences for statistical analysis can be at least 1, 5, 10, 20, 50 or 100.

### Recombinant Expression Cassettes

The present disclosure further provides recombinant expression cassettes comprising a nucleic acid for modulating protein membrane content, preferably designed for increasing the activity of FADL and decreasing the activity of OmpF. A nucleic acid sequence coding for the desired polynucleotide of the present disclosure, for example a cDNA or a genomic sequence encoding a polypeptide long enough to code for an active protein of the present disclosure, can be used to construct a recombinant expression cassette which can be introduced into the desired bacterial host cell. A recombinant expression cassette will typically comprise a polynucleotide operably linked to transcriptional initiation regulatory sequences which will direct the transcription of the polynucleotide in the intended host cell. For example, organism expression vectors may include (1) a cloned phospholipid synthase gene under the transcriptional control of 5′ and 3′ regulatory sequences an FADL or OmpF (2) a dominant selectable marker. Such organism expression vectors may also contain, if desired, a promoter regulatory region (e.g., one conferring inducible or constitutive, environmentally- or developmentally-regulated, or cell- or tissue-specific/selective expression), a transcription initiation start site, a ribosome binding site, an RNA processing signal, a transcription termination site and/or a polyadenylation signal.

### Promoters, Terminators, Introns

An organism promoter fragment can be employed which will direct expression of a polynucleotide of the present disclosure in essentially all tissues of a regenerated organism. Such promoters are referred to herein as “constitutive” promoters and are active under most environmental conditions and states of development or cell differentiation. Examples of constitutive promoters include the 1′- or 2′-promoter derived from T-DNA of Agrobacterium tumefaciens, the Smas promoter, the cinnamyl alcohol dehydrogenase promoter (U.S. Pat. No. 5,683,439), the Nos promoter the GRP1-8 promoter, the 35S promoter from cauliflower mosaic virus (CaMV), as described in Odell, et al., (1985) Nature 313:810-2; rice actin (McElroy, et al., (1990) Organism Cell 163-171); ubiquitin (Christensen, et al., (1992) Organism Mol. Biol. 12:619-632 and Christensen, et al., (1992) Organism Mol. Biol. 18:675-89); pEMU (Last, et al., (1991) Theor. Appl. Genet. 81:581-8); MAS (Velten, et al., (1984) EMBO J. 3:2723-30) and maize H3 histone (Lepetit, et al., (1992) Mol. Gen. Genet. 231:276-85 and Atanassvoa, et al., (1992) Organism Journal 2(3):291-300); ALS promoter, as described in PCT Application Number WO 1996/30530 and other transcription initiation regions from various organism genes known to those of skill. For the present disclosure ubiquitin is the preferred promoter for expression in monocot organisms.

Alternatively, the organism promoter can direct expression of a polynucleotide of the present disclosure in a specific tissue or may be otherwise under more precise environmental or developmental control. Such promoters may be “inducible” promoters. Environmental conditions that may affect transcription by inducible promoters include pathogen attack, anaerobic conditions or the presence of light. Examples of inducible promoters are the Adh1 promoter, which is inducible by hypoxia or cold stress, the Hsp70 promoter, which is inducible by heat stress and the PPDK promoter, which is inducible by light. Diurnal promoters that are active at different times during the circadian rhythm are also known (US Patent Application Publication Number 2011/0167517, incorporated herein by reference). Examples of promoters under developmental control include promoters that initiate transcription only, or preferentially, in certain tissues, such as leaves, roots, fruit, seeds or flowers. The operation of a promoter may also vary depending on its location in the genome. Thus, an inducible promoter may become fully or partially constitutive in certain locations.

If polypeptide expression is desired, it is generally desirable to include a polyadenylation region at the 3′-end of a polynucleotide coding region. The polyadenylation region can be derived from a variety of organism genes, or from T-DNA. The 3′ end sequence to be added can be derived from, for example, the nopaline synthase or octopine synthase genes or alternatively from another organism gene or less preferably from any other eukaryotic gene. Examples of such regulatory elements include, but are not limited to, 3′ termination and/or polyadenylation regions such as those of the Agrobacterium tumefaciens nopaline synthase (nos) gene (Bevan, et al., (1983) Nucleic Acids Res. 12:369-85); the potato proteinase inhibitor II (PINII) gene (Keil, et al., (1986) Nucleic Acids Res. 14:5641-50 and An, et al., (1989) Organism Cell 1:115-22) and the CaMV 19S gene (Mogen, et al., (1990) Organism Cell 2:1261-72).

### Signal Peptide Sequences

Organism signal sequences, including, but not limited to, signal-peptide encoding DNA/RNA sequences which target proteins to the extracellular matrix of the organism cell (Dratewka-Kos, et al., (1989) J. Biol. Chem. 264:4896-900), such as the Nicotiana plumbaginifolia extension gene (DeLoose, et al., (1991) Gene 99:95-100); signal peptides which target proteins to the vacuole, such as the sweet potato sporamin gene (Matsuka, et al., (1991) Proc. Natl. Acad. Sci. USA 88:834) and the barley lectin gene (Wilkins, et al., (1990) Organism Cell, 2:301-13); signal peptides which cause proteins to be secreted, such as that of PRIb (Lind, et al., (1992) Organism Mol. Biol. 18:47-53) or the barley alpha amylase (BAA) (Rahmatullah, et al., (1989) Organism Mol. Biol. 12:119) or signal peptides which target proteins to the plastids such as that of rapeseed enoyl-Acp reductase (Verwaert, et al., (1994) Organism Mol. Biol. 26:189-202) are useful in the disclosure.

### Markers

The vector comprising the sequences from a polynucleotide of the present disclosure will typically comprise a marker gene, which confers a selectable phenotype on organism cells. The selectable marker gene may encode antibiotic resistance, with suitable genes including genes coding for resistance to the antibiotic spectinomycin (e.g., the aada gene), the streptomycin phosphotransferase (SPT) gene coding for streptomycin resistance, the neomycin phosphotransferase (NPTII) gene encoding kanamycin or geneticin resistance, the hygromycin phosphotransferase (HPT) gene coding for hygromycin resistance gene.

Constructs described herein may comprise a polynucleotide of interest encoding a reporter or marker product. Examples of suitable reporter polynucleotides known in the art can be found in, for example, Jefferson, et al., (1991) in Organism Molecular Biology Manual, ed. Gelvin, et al., (Kluwer Academic Publishers), pp. 1-33; DeWet, et al. (1987) Mol. Cell. Biol. 7:725-737; Goff, et al., (1990) EMBO J. 9:2517-2522; Kain, et al., (1995) Bio Techniques 19:650-655 and Chiu, et al., (1996) Current Biology 6:325-330. In certain embodiments, the polynucleotide of interest encodes a selectable reporter. These can include polynucleotides that confer antibiotic resistance.

In some embodiments, the expression cassettes disclosed herein comprise a polynucleotide of interest encoding scorable or screenable markers, where presence of the polynucleotide produces a measurable product. Examples include a β-glucuronidase, or uidA gene (GUS), which encodes an enzyme for which various chromogenic substrates are known (for example, U.S. Pat. Nos. 5,268,463 and 5,599,670); chloramphenicol acetyl transferase and alkaline phosphatase. Other screenable markers include the anthocyanin/flavonoid polynucleotides including, for example, a R-locus polynucleotide, which encodes a product that regulates the production of anthocyanin pigments (red color) in organism tissues, the genes which control biosynthesis of flavonoid pigments, such as the maize C1 and C2, the B gene, the p1 gene and the bronze locus genes, among others. Further examples of suitable markers encoded by polynucleotides of interest include the cyan fluorescent protein (CYP) gene, the yellow fluorescent protein gene, a lux gene, which encodes a luciferase, the presence of which may be detected using, for example, X-ray film, scintillation counting, fluorescent spectrophotometry, low-light video cameras, photon counting cameras or multiwell luminometry, a green fluorescent protein (GFP) and DsRed2 (Clontechniques, 2001) where organism cells transformed with the marker gene are red in color, and thus visually selectable. Additional examples include a p-lactamase gene encoding an enzyme for which various chromogenic substrates are known (e.g., PADAC, a chromogenic cephalosporin), a xylE gene encoding a catechol dioxygenase that can convert chromogenic catechols, an alpha amylase gene and a tyrosinase gene encoding an enzyme capable of oxidizing tyrosine to DOPA and dopaquinone, which in turn condenses to form the easily detectable compound melanin. The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as beta-galactosidase and fluorescent proteins such as green fluorescent protein (GFP) (Su, et al., (2004) Biotechnol Bioeng 85:610-9 and Fetter, et al., (2004) Organism Cell 16:215-28), cyan florescent protein (CYP) (Bolte, et al., (2004) J. Cell Science 117:943-54 and Kato, et al., (2002) Organism Physiol 129:913-42) and yellow florescent protein (PhiYFP™ from Evrogen, see, Bolte, et al., (2004) J. Cell Science 117:943-54). For additional selectable markers, see generally, Yarranton, (1992) Curry. Opin. Biotech. 3:506-511; Christopherson, et al., (1992) Proc. Natl. Acad. Sci. USA 89:6314-6318; Yao, et al., (1992) Cell 71:63-72; Reznikoff, (1992) Mol. Microbiol. 6:2419-2422; Barkley, et al., (1980) in The Operon, pp. 177-220; Hu, et al., (1987) Cell 48:555-566; Brown, et al., (1987) Cell 49:603-612; Figge, et al., (1988) Cell 52:713-722; Deuschle, et al., (1989) Proc. Natl. Acad. Aci. USA 86:5400-5404; Fuerst, et al., (1989) Proc. Natl. Acad. Sci. USA 86:2549-2553; Deuschle, et al., (1990) Science 248:480-483; Gossen, (1993) Ph.D. Thesis, University of Heidelberg; Reines, et al., (1993) Proc. Natl. Acad. Sci. USA 90:1917-1921; Labow, et al., (1990) Mol. Cell. Biol. 10:3343-3356; Zambretti, et al., (1992) Proc. Natl. Acad. Sci. USA 89:3952-3956; Bairn, et al., (1991) Proc. Natl. Acad. Sci. USA 88:5072-5076; Wyborski, et al., (1991) Nucleic Acids Res. 19:4647-4653; Hillenand-Wissman, (1989) Topics Mol. Struc. Biol. 10:143-162; Degenkolb, et al., (1991) Antimicrob. Agents Chemother. 35:1591-1595; Kleinschnidt, et al., (1988) Biochemistry 27:1094-1104; Bonin, (1993) Ph.D. Thesis, University of Heidelberg; Gossen, et al., (1992) Proc. Natl. Acad. Sci. USA 89:5547-5551; Oliva, et al., (1992) Antimicrob. Agents Chemother. 36:913-919; Hlavka, et al., (1985) Handbook of Experimental Pharmacology, Vol. 78 (Springer-Verlag, Berlin); Gill, et al., (1988) Nature 334:721-724. Such disclosures are herein incorporated by reference. The above list of selectable marker genes is not meant to be limiting. Any selectable marker gene can be used in the compositions and methods disclosed herein.

### Expression in Prokaryotes

According to the invention, prokaryotic bacterial strains are modified for phospholipid membrane content. Prokaryotic cells may also be used to increase and produce vectors for expression constructs. Prokaryotes most frequently are represented by various strains of E. coli; however, other microbial strains may also be used. Commonly used prokaryotic control sequences which are defined herein to include promoters for transcription initiation, optionally with an operator, along with ribosome binding site sequences, include such commonly used promoters as the beta lactamase (penicillinase) and lactose (lac) promoter systems (Chang, et al., (1977) Nature 198:1056), the tryptophan (trp) promoter system (Goeddel, et al., (1980) Nucleic Acids Res. 8:4057) and the lambda derived P L promoter and N-gene ribosome binding site (Shimatake, et al., (1981) Nature 292:128). The inclusion of selection markers in DNA vectors transfected in E. coli is also useful. Examples of such markers include genes specifying resistance to ampicillin, tetracycline, or chloramphenicol.

The vector is selected to allow introduction of the gene of interest into the appropriate host cell. Bacterial vectors are typically of plasmid or phage origin. Appropriate bacterial cells are infected with phage vector particles or transfected with naked phage vector DNA. If a plasmid vector is used, the bacterial cells are transfected with the plasmid vector DNA. Expression systems for expressing a protein of the present invention are available using Bacillus sp. and Salmonella (Palva, et al., (1983) Gene 22:229-35; Mosbach, et al., (1983) Nature 302:543-5). The pGEX-4T-1 plasmid vector from Pharmacia is the preferred E. coli expression vector for the present invention.

### Reducing the Activity of a Polypeptide/Gene

In some embodiments, the activity of a transporter protein/gene is reduced or inactivated. A transporter with reduced activity or one which is inactivated can be produced by effecting deletion, insertion, inversion, substitution, derivatization, and/or or any other change of amino acids at the amino acid level in order to deactivate the transporter protein. Alternatively, inactivation can occur at the nucleic acid level by genetic modification (also referred to herein as mutation). Genetic modification includes deletion, insertion, inversion, substitution, derivatization, and/or any other change known to those skilled in the art of one or more nucleotides in a gene encoding the transporter protein such that the genetically modified nucleic acid does not encode an active transporter protein. In some embodiments, no transporter protein is produced at all. In some embodiments, the encoded transporter protein is truncated.

Specific inactivation may be obtained by random mutation followed by screening or selection, or, where the gene sequence is known, by direct intervention by molecular biology methods known to those skilled in the art.

In some embodiments inactivation is achieved by 1) deleting coding regions and/or regulatory (promoter) regions, 2) inserting exogenous nucleic acid sequences into coding regions and/regulatory (promoter) regions, and 3) altering coding regions and/or regulatory (promoter) regions (for example, by making DNA base pair changes).

In one embodiment, such a deletion comprises Cre-loxP gene knockout. One embodiment is a conditionally inactivated transporter protein, which can be produced, for example, using inducible antisense or anti-parallel loxP sites bracketing the structural gene encoding such a transporter protein so that the Cre recombinase flips the gene between active and inactive forms. In one embodiment, a transporter protein gene is inactivated via a Saccharomyces cerevisiae FLP-FRT recombinase system. In another embodiment, the gene inactivation may be obtained by RED/ET methods using kits and other reagents sold by Gene Bridges (Gene Bridges GmbH. Dresden. Germany,

More particularly as to the latter method, use of Red/ET recombination, is known to those of ordinary skill in the art and described in U.S. Pat. Nos. 6,355,412 and 6,509,156, issued to Stewart et al. and incorporated by reference herein for its teachings of this method. Material and kits for such method are available from Gene Bridges (Gene Bridges GmbH, Dresden, Germany, world wide web at genebridges.com, and the method may proceed by following the manufacturer's instructions. The method involves replacement of the target gene by a selectable marker via homologous recombination performed by the recombinase from λ-phage. The host microorganism expressing X-red recombinase is transformed with a linear DNA product coding for a selectable marker flanked by the terminal regions generally about 50 bp, and alternatively up to about 300 bp) homologous with the target gene. The marker could then be removed by another recombination step performed by a plasmid vector carrying the FLP-recombinase, or another recombinase, such as Cre.

Another example of a gene disruption includes a knockout microorganism in which the functions of genes on the genome are disrupted. Such a transformant can be prepared generally by using a gene targeting recombination method (gene targeting method: for example, Methods in Enzymology 225: 803-890, 1993) known in the art, for example, by homologous recombination. The homologous recombination method can be performed by inserting a target DNA into the sequence homologous to the sequence on the genome, introducing the DNA fragment into a cell, and allowing the cell to cause homologous recombination. In the introducing into the genome, a strain in which homologous recombination took place can be easily screened by using a DNA fragment in which a target DNA is ligated to a drug resistant gene. Alternatively, it is also possible to insert a DNA fragment in which a drug resistant gene is ligated to a gene to be lethal under specific conditions by homologous recombination thereafter substituting the drug resistant gene with the gene to be lethal under specific conditions. Furthermore, a method using group II intron found in lactobacillus (Guo et. al., Science 21; 289 (5478): 452-7 (2000)) and a genome processing method such as TALEN technology and CRISPR technology can be used.

Additional methods for decreasing or eliminating the expression of endogenous genes in bacteria are also known in the art and can be similarly applied to the instant invention. These methods include other forms of mutagenesis, such as ethyl methanesulfonate-induced mutagenesis, deletion mutagenesis, and fast neutron deletion mutagenesis used in a reverse genetics sense (with PCR) to identify cell lines in which the endogenous gene has been deleted. For examples of these methods see, Ohshima, et al., (1998) Virology 243:472-481; Okubara, et al., (1994) Genetics 137:867-874; and Quesada, et al., (2000) Genetics 154:421-436; each of which is herein incorporated by reference. Mutations that impact gene expression or that interfere with the function of the encoded protein are well known in the art. Insertional mutations in gene exons usually result in null-mutants. Mutations in conserved residues are particularly effective in inhibiting the activity of the encoded protein. Conserved residues of bacteria polypeptides suitable for mutagenesis with the goal to eliminate activity have been described. Such mutants can be isolated according to well-known procedures, and mutations in different loci can be stacked by genetic crossing. See, for example, Gruis, et al., (2002) Plant Cell 14:2863-2882.

The invention encompasses additional methods for reducing or eliminating the activity of one or more polypeptides. Examples of other methods for altering or mutating a genomic nucleotide sequence in a bacterium are known in the art and include, but are not limited to, the use of RNA: DNA vectors, RNA: DNA mutational vectors, RNA: DNA repair vectors, mixed-duplex oligonucleotides, self-complementary RNA: DNA oligonucleotides, and recombinogenic oligonucleobases. Such vectors and methods of use are known in the art. See, for example, U.S. Pat. Nos. 5,565,350; 5,731,181; 5,756,325; 5,760,012; 5,795,972; and 5,871,984; each of which are herein incorporated by reference. See also, WO 98/49350, WO 99/07865, WO 99/25821, and Beetham, et al., (1999) Proc. Natl. Acad. Sci. USA 96:8774-8778; each of which is herein incorporated by reference.

### IV. Methods for Producing Biorenewables

Accordingly, the present invention provides methods for the production of fatty acids from pure sugars, and other compounds. A variety of culture methodologies may be applied to the modified strains described herein. For example, large-scale production of a specific product made possible by the modified strains described herein may be accomplished by both batch and/or continuous culture methodologies.

A classical batch culturing method is a closed system where the composition of the media is set at the beginning of the culture and not subject to external alterations during the culturing process. Thus, at the beginning of the culturing process the medium is inoculated with the desired strain and growth or metabolic activity is permitted to occur adding nothing to the system. Typically, however, a “batch” culture is batch with respect to the addition of carbon source and attempts are often made at controlling factors such as pH and oxygen concentration. In batch systems, the composition of the system changes constantly up to the time the culture is terminated. Within batch cultures, strain cells moderate through a static lag phase to a high growth log phase and finally to a stationary phase where growth rate is diminished or halted. If untreated, cells in the stationary phase will eventually die. Cells in log phase are often responsible for the bulk of production of end product or intermediate in some systems. Stationary or post-exponential phase production can be obtained in other systems.

A variation on the standard batch system is the Fed-Batch system. Fed-Batch culture processes are also suitable and comprise a typical batch system with the exception that the substrate is added in increments as the culture progresses. Fed-Batch systems are useful when catabolite repression is apt to inhibit the metabolism of the cells and where it is desirable to have limited amounts of substrate in the medium. Measurement of the actual substrate concentration in Fed-Batch systems is difficult and is therefore estimated on the basis of the changes of measurable factors such as pH, dissolved oxygen and the partial pressure of waste gases such as CO₂. Batch and Fed-Batch culturing methods are common and well known in the art and examples may be found in Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol., 36:227 (1992) each of which is incorporated by reference herein for its teachings regarding the same.

Continuous cultures can also be used. Continuous cultures are open systems where a defined culture medium is added continuously to a bioreactor and an equal amount of conditioned medium is removed simultaneously for processing. Continuous cultures generally maintain the cells at a constant high liquid phase density where cells are primarily in log phase growth. Alternatively, continuous culture can be practiced with immobilized cells where carbon and nutrients are continuously added and valuable products, by-products, and waste products are continuously removed from the cell mass. Cell immobilization may be performed using a wide range of solid supports composed of natural and/or synthetic materials.

Continuous or semi-continuous culture allows for the modulation of one factor or any number of factors that affect cell growth or end product concentration. For example, one method maintains a limiting nutrient such as the carbon source or nitrogen level at a fixed rate and allows all other parameters to moderate. In other systems, a number of factors affecting growth can be altered continuously while the cell concentration, measured by medium turbidity, is kept constant. Continuous systems strive to maintain steady state growth conditions and thus the cell loss due to medium being drawn off must be balanced against the cell growth rate in the culture. Methods of modulating nutrients and growth factors for continuous culture processes, as well as techniques for maximizing the rate of product formation, are well known in the art of industrial microbiology and a variety of methods are detailed by Brock Biology of Microorganisms, 8th edition, Prentice Hall, UpperSaddle River, N.J. (1997) which is incorporated by reference herein for its teachings regarding the same.

The strains of the invention are typically grown in a simple mineral medium (NMS) supplemented with salt and carbonate buffer. In some embodiments, in batch culture, optimal growth occurred at pH 9.0-9.5 and with 0.75% NaCl. The invention also provides for a kit comprising an isolated or recombinant bacterium of the invention as described above. This kit optionally provides instructions for use, such as, for example, instructions for producing fatty acids in accordance with the methods and processes described herein.

All publications, patents and patent applications identified herein are incorporated by reference, as though set forth herein in full. The invention being thus described, it will be apparent to those skilled in the art that the same may be varied in many ways without departing from the spirit and scope of the invention. Such variations are included within the scope of the following claims.

The invention is further illustrated by the following specific examples which are not intended in any way to limit the scope of the invention.

### Example 1

Construction of microbial biocatalysts for the production of biorenewables at economically viable yields and titers is frequently hampered by product toxicity. Membrane damage is often deemed as the principal mechanism of this toxicity, particularly in regard to decreased membrane integrity. Previous studies have attempted to engineer the membrane with the goal of increasing membrane integrity. However, most of these works focused on engineering of phospholipids and efforts to identify membrane proteins that can be targeted to improve fatty acid production have been unsuccessful.

### Results

Here we show that deletion of outer membrane protein ompF significantly increased membrane integrity, fatty acid tolerance and fatty acid production, possibly due to prevention of re-entry of short chain fatty acids. In contrast, deletion of fadL resulted in significantly decreased membrane integrity and fatty acid production. Consistently, increased expression of fadL remarkably increased membrane integrity and fatty acid tolerance while also increasing the final fatty acid titer. This 34% increase in the final fatty acid titer was possibly due to increased membrane lipid biosynthesis. Tuning of fadL expression showed that there is a positive relationship between fadL abundance and fatty acid production. Combinatorial deletion of ompF and increased expression of fadL were found to have an additive role in increasing membrane integrity, and was associated with a 53% increase the fatty acid titer, to 2.3 g/L.

### Conclusions

These results emphasize the importance of membrane proteins for maintaining membrane integrity and production of biorenewables, such as fatty acids, which expands the targets for membrane engineering.

### Background

Construction of microbial cell factories for production of biorenewable fuels and chemicals is a promising alternative to current petroleum-driven industries [1, 2]. A variety of microorganisms have been engineered for production of bulk chemicals, biofuels and high-value, fine chemicals [3-7]. However, performance of some biocatalysts can be restricted by various detrimental effects, including toxicity of the product or components of the feedstock [8, 9]. A variety of adverse effects could be the cause of this toxicity, e.g. intracellular acidification; DNA, RNA, protein and membrane damage [10]. Among these, membrane damage has been recognized as a frequent problem [11-15]. Membrane damage can be compared to a reaction vessel that is vulnerable to corrosion by its contents. In this scenario, a typical response would be to change the composition of the reaction vessel in order to increase resistance to corrosion. For microbial biocatalysts, the composition, function and physical properties of the membrane can be altered through targeted, rational genetic manipulation. Such genetic manipulation is consistent with Cameron and Tong's fifth application of cellular and metabolic engineering, “modification of cell properties” [16]. When enzymes, transporters and regulators are involved in this membrane engineering, it is also consistent with Bailey's 1991 definition of metabolic engineering as “the improvement of cellular activities by manipulation of enzymatic, transport, and regulatory function of the cell with the use of recombinant DNA technology” [17].

This work focuses on membrane engineering to improve production of fatty acids, an attractive class of biorenewable chemicals which can be catalyzed to a variety of products with a large potential market, e.g. alkanes, olefins, esters, fatty aldehydes, and fatty alcohols [18-22]. Unfortunately, these fatty acids have been reported to cause a decrease in membrane integrity of E. coli during both exogenous challenge and endogenous production [14]. Engineering of membrane phospholipids has proven as a powerful tool in addressing membrane integrity. Decreasing incorporation of medium-chain fatty acids into the membrane increased the average membrane lipid length, decreased the toxicity of fatty acids and increased fatty acid (C12-C14) production in rich medium from 0.60 to 1.36 g/L [23]. Expression of a thioesterase from Geobacillus sp Y412MC10 that prevents medium-chain unsaturated acyl-ACPs from being incorporated into the phospholipids was shown to increase membrane integrity during fatty acid production, but there was no increase in fatty acid (C8-C14) production after 24 hours in rich medium, with titers of 0.65 g/L observed with and without expression of the secondary thioesterase [24]. Both of these works demonstrate the feasibility of engineering the membrane lipid composition in order to increase membrane integrity and possibly enhance fatty acid tolerance and production [23, 24].

As efforts continue to increase the membrane integrity during production of membrane-damaging compounds, it becomes increasingly important to provide a sufficient route of product export. Several studies have shown that increasing the expression of transporters can increase production of inhibitory compounds, such as valine [25] and limonene [8]. With the goal of using this strategy to improve fatty acid production, sixteen possible fatty acid transporters were characterized for their role in fatty acid tolerance and production [26]. This previous study identified several transporters that increased fatty acid tolerance when their expression was increased, but did not identify any such transporters that increased fatty acid production.

The transporters OmpF and FadL were part of the previous study. The OmpF protein exists as a trimer in the outer membrane and participates in the transport of sugars, ions, antibiotics and proteins across the outer membrane [27, 28]. FadL is an outer membrane ligand gated channel that functions in the uptake of exogenous long-chain fatty acids (LCFA), [29, 30], especially palmitic acid (C16:0) and oleic acid (C18:1), yet shows no binding to short-chain fatty acids (SCFA, <C10) [31]. Even though the previous characterization observed that deletion of ompF and fadL had no impact on fatty acid production [26], several other reports related to these two transporters (Table 1) motivated the further exploration of their role in fatty acid tolerance and production described here.

TABLE 1
Previous reports of the role of OmpF and FadL in tolerance of membrane-damaging compounds

| | | | |
|---|---|---|---|
| Compound | Condition | Result | Reference |

OmpF, outer membrane porin F

| | | | |
|---|---|---|---|
| C₈-C₁₄ fatty acids | Production of ~1 g/L fatty acids | Deletion of ompF from a derivative of MG1655 had no impact on cell | [26] |
| | during growth in LB with glycerol, | viability or membrane integrity | |
| | 37° C. | | |
| Octanoic acid (C8) | Challenge with up to 20 mM C8 in | Deletion of ompF from BW25113 decreased sensitivity to C8, and | [32] |
| | minimal media with glucose, | increased expression of ompF increased sensitivity to C8. Sensitivity was | |
| | tryptone and yeast extract at pH | assessed via the maximum OD. Deletion of ompF decreased the magnitude | |
| | 7.0 and 37° C. | of intracellular acidification | |
| Phenylpropanoids | Challenge with 1 g/L rutin, | Increased expression of ompF in BL21 increased the maximum specific | [33] |
| | naringenin or resveratrol in M9 | growth rate during challenge. Decreased growth rate during challenge was | |
| | medium with casamino acids and | observed when ompF expression was decreased | |
| | glucose at 30° C. | | |

FadL, long-chain fatty acid outer membrane porin

| | | | |
|---|---|---|---|
| C₈-C₁₄ fatty acids | Production of ~1 g/L fatty acids | Deletion of fadL from a derivative of MG1655 had no impact on cell | [26] |
| | during growth in LB with glycerol | viability or membrane integrity | |
| | at 37° C. | | |
| Palmitic and ω- | Addition of 1 mM palmitic acid in | Increased expression of fadL increased conversion of palmitic acid to ω- | [34] |
| hydroxy palmitic acids | potassium phosphate buffer with | hydroxy palmitic acid. The increase was smaller in the presence of | |
| | glucose or glycerol, 30° C. | glycerol than glucose | |
| Phenol | Challenge with phenol at 50-75% | Deletion of fadL from BW25113 had no impact on survival | [37] |
| | of the MIC in LB at 37° C. | | |
| Octane | Addition of ~20 vol % octane in | Deletion of fadL from a BW25113 derivative abolished conversion of | [35] |
| | LB at 37° C. | octane to octanol, octanal and octanoic acid | |
| Hexane | Challenge with 10 vol % hexane in | Deletion of fadL from BW25113 increased survival, as assessed by OD | [35] |
| | LB at 37° C. | | |
| Phenylpropanoids | Challenge with 1 g/L rutin, | Increased expression of fadL in BL21 increased the maximum specific | [33] |
| | naringenin or resveratrol in | growth rate during challenge. Decreased growth rate during challenge was | |
| | minimal medium with casamino | observed when fadL expression was decreased | |
| | acids and glucose at 30° C. | | |

Two 2015 publications directly implicated OmpF in tolerance of exogenously supplied inhibitors, though in one case OmpF played a protective role and in the other it played a damaging role. Most relevant to our goal of improving fatty acid production is the demonstration that deletion of ompF dampened octanoic acid toxicity, with evidence that this deletion of ompF reduced SCFA entry into cells [32] (Table 1). This reduced entry of SCFA into cells was assessed by measuring the decrease in intracellular pH during challenge with exogenously supplied octanoic acid. Contrastingly, OmpF was found to be directly related to tolerance of three exogenously provided phenylpropanoids: rutin, naringenin and resveratrol [33]. Specifically, strains with increased expression of OmpF showed increased tolerance to these compounds and strains with decreased expression of OmpF showed decreased tolerance, leading to the proposition that OmpF participates in the removal of phenylpropanoids from the cell interior. Thus, OmpF showed a negative role in SCFA tolerance and a positive role in phenylpropanoid tolerance.

There are also reports of FadL being involved in fatty acid production and tolerance to some inhibitors (Table 1). Increased expression of fadL resulted in increased conversion of exogenously supplied palmitic acid to ω-hydroxy palmitic acid [34]. This improved organism performance was attributed to increased uptake of palmitic acid, as data indicated that FadL was not involved in export of the hydroxylated product. Similarly, FadL seemed to play a crucial role in the import of octane for production of octanol, octanal and octanoic acid [35]. Specifically, production of these compounds from exogenously supplied octane was abolished when fadL was deleted. However, it was noted that this deletion of fadL increased survival during challenge with hexane, with the conclusion that FadL was the main route of hexane entry into the cell [35]. The phenylpropanoid studies described above also noted that FadL abundance was directly related to tolerance of exogenously supplied rutin, naringenin and resveratrol, the same trend was observed for OmpF, with the interpretation that FadL was involved in repairing membrane damage caused by these compounds [33]. However, even though phenol toxicity is often attributed to membrane damage [36], deletion of fadL had no impact on survival during phenol challenge [37]. Thus, FadL appears to be important to the uptake of some fatty acids and alkanes, provides protection from the inhibitory effects of phenylpropanoids, provides entry to some harmful alkanes and yet possibly plays no role in repairing the membrane damage caused by phenol.

Here we have taken another look at the role of OmpF and FadL in fatty acid tolerance and production, with the conclusion that OmpF and FadL have opposite effects. Specifically, fatty acid tolerance, fatty acid production and membrane integrity were all increased when ompF was deleted or when expression of fadL was increased. Concurrent utilization of these two engineering strategies enabled a roughly 50% increase in production of fatty acids (primarily C14, C16:1 and C16), resulting in a final titer of 2.3 g/L. Although we employed a thioesterase specific for LCFA (C14-C16), some SCFAs (e.g. C8 and C10) were also produced. We propose that deletion of ompF prevents re-entry of the SCFA and their corresponding toxic effects. Contrastingly, it seems that FadL may enable the recapture of some of the LCFA for use in membrane biosynthesis and repair.

### Methods

Strains and Plasmids: All plasmids and strains used in this study are listed in Table 2.

TABLE 2
Strains and plasmids used in this study.

| Strains/plasmids | Genetic characteristics | Source |
|---|---|---|
| Strains | | |
| MG1655 | Wild type E. coli K-12 strain | Lab |
| | | collection |
| ΔompF | MG1655, ΔompF | This study |
| ΔfadD | MG1655, ΔfadD | This study |
| ΔfadL | MG1655, ΔfadL | This study |
| Pla-empty | MG1655, pACYC184-Kan | This study |
| Pla-fadL | MG1655, pACYC184-Kan-fadL | This study |
| Gen-empty | MG1655, ldhA::FRT-cat-FRT | This study |
| Gen-fadL | MG1655, ldhA::FRT-cat-FRT, fadL | This study |
| M1-12-fadL | MG1655, FRT-cat-FRT, M1-12-fadL | This study |
| M1-37-fadL | MG1655, FRT-cat-FRT, M1-37-fadL | This study |
| M1-46-fadL | MG1655, FRT-cat-FRT, M1-46-fadL | This study |
| M1-93-fadL | MG1655, FRT-cat-FRT, M1-93-fadL | This study |
| ΔompF + Pla-empty | MG1655, ΔompF, pACYC184- | This study |
| | Kan | |
| ΔompF + Pla-fadL | MG1655, ΔompF, pACYC184- | This study |
| | Kan-fadL | |
| Plasmids | | |
| pACYC184-Kan | p15A, pACYC184, Kan^{r} | This study |
| pACYC184-Kan- | pACYC184-Kan harboring fadL, | This study |
| fadL | Kan^{r} | |
| pXZ18Z (TE) | pTrc99a-Ricinus communis | [42] |
| | thioesterase-fabZ, Amp^{r} | |

One-step recombination method (FLP-FRT) was employed to perform genetic modifications [38]. E. coli K-12 MG1655 was employed as the host strain. GenBank Accession No. U00096.2. For modulating expression of fadL (Genbank Accession Sequence: NC_000913.3 (2461306.2462646)) the FRT-cat-FRT selection marker linked with four different promoters (M1-12, M1-37, M1-46, M1-93) [6, 39, 40] with varying strengths was employed to regulate expression of the original fadL gene, yielding engineered strains M1-12-fadL, M1-37-fadL, M1-46-fadL and M1-93-fadL, respectively.

For increasing expression of fadL, two different strategies were employed. First, the low-copy plasmid pACYC184-Kan-fadL, which harbors the native promoter, open reading frame (ORF), and terminator of fadL was transformed to MG1655, resulting in Pla-fadL. MG1655 with empty pACYC184-Kan served as the corresponding control (Pla-empty). Second, for increased expression of fadL from the chromosome, a second copy of the fadL gene was inserted into the MG1655 genome at the ldhA site, resulting in Gen-fadL. The ldhA gene was also deleted from MG1655 to generate strain Gen-empty, which serves as a control for strain Gen-fadL. Selection of ldhA as the integration site was motivated by previous reports [41].

The pXZ18Z plasmid [42] harboring a thioesterase from Ricinus communis and the E. coli 3-hydroxyacyl-ACP dehydratase (fabZ) was used for long-chain fatty acid (LCFA) production. When necessary, ampicillin, kanamycin and chloramphenicol were used at final concentrations of 100 mg/L, 50 mg/L and 34 mg/L, respectively.

2.2 Strain Tolerance Characterization: Octanoic acid tolerance was characterized in 50 ml MOPS defined minimal medium with 2.0% (wt/v) dextrose and 10 mM octanoic acid (1.44 g/L) in 250 ml baffled flasks at 220 rpm and initial pH at 7.0, 30° C. MOPS media contains the following: 8.37 g/L 3-(N-morpholino)propanesulfonic acid (MOPS), 0.72 g/L tricine, 2.92 g/L NaCl, 0.51 g/L NH4Cl, 1.6 g/L KOH, 50 mg/L MgCl2, 48 mg/L K2SO4, 348 mg/L K2HPO4, 0.215 mg/L Na2SeO3, 0.303 mg/L Na2MoO4.2H2O, 0.17 mg/L ZnCl2, 2.5 μg/L FeCl2.4H2O, 0.092 μg/L CaCl2.2H2O, 0.031 μg/L H3BO3, 0.020 μg/L MnCl2.4H2O, 0.0090 μg/L CoCl2.4H2O, and 0.0020 μg/L CuCl2.4H2O [43, 44]. Specific growth rate μ (h-1) was calculated by fitting the equation OD=OD₀e^{μt} over the duration of the exponential growth phase. OD was measured at 550 nm and all estimated μ values had an R² of at least 0.95 [45]. Dry cell weight (DCW) was calculated from the optical density at 550 nm (1 OD550=0.333 g DCW/L).

Membrane Integrity Characterization: Cells were centrifuged, washed twice, and then resuspended in PBS buffer (pH 7.0) at a final OD550 of ˜1. One hundred microliter (100 μl) of this suspension was mixed with 900 μl of PBS buffer and SYTOX Green (Invitrogen) was added to a final concentration of 5.0 μM. After resting at room temperature for 15 minutes, cells were analyzed by a BD Biosciences FACSCanto II flow cytometer equipped with standard factory-installed 488 nm excitation laser, signal collection optics, and fluorescence emission filter configuration. Instrument sheath fluid was filtered (0.22 μm) PBS buffer. Green fluorescence from stained cells was collected in the FL1 channel (525/50 nm). Forward scatter (FSC), side scatter (SSC), and FL1 (Green) parameters were collected as logarithmic signals. All data collections were performed at low flow rate setting (˜12 μl/min) and cell concentrations were such that the event rate was below 5,000 events/s. All samples were analyzed immediately after staining. Background noise and small debris was eliminated from data collection via a side scatter signal threshold that was established by examining samples containing only SYTOX Green staining buffer. Bacteria in SYTOX Green-stained samples were readily identified on the basis of FSC and SSC signals and an appropriate “Cell” gate was drawn to limit FL1 analysis to bacteria and exclude non-cell events. A minimum of 20,000 cell-gated events were collected for each sample. Green fluorescence data for these “cell” events were plotted as histograms showing the signal distribution of bacteria in the sample [14]. Flow cytometry data for this work is available via Flow Repository (https://flowrepository.org) (FR-FCM-ZY2B).

Membrane Lipid Composition Characterization: The membrane lipids were extracted by using the Bligh and Dyer method with minor modifications [14, 46]. Cells were centrifuged, washed twice with cold double-distilled water (ddH2O), resuspended in 1.4 ml methanol and transferred to a new glass tube. Ten μl of 1 μg/μl pentadecanoic acid (C15:0) dissolved in ethanol was added as internal standard. Then, samples were sonicated, incubated at 70° C. for 15 min and centrifuged at 5,000×g for 5 min. The supernatant was transferred to a new glass tube and the cell pellet was resuspended in 0.75 ml of chloroform, shaken at 37° C., 150 rpm for 5 min. Transferred supernatant and pellet suspension were combined, vortexed for 1 min and centrifuged at 5,000×g for 2 min. The bottom phase was transferred to a new glass tube and dried under nitrogen gas. Two ml of methanol: sulfuric acid (98:2 v/v) mixture was added and the mixture was vortexed and incubated at 80° C. for 30 min. Finally, 2 ml of 0.9% (wt/v) sodium chloride (NaCl) and 1 ml of hexane were added, vortexed and centrifuged at 2,000×g for 2 min. The top hexane layer was then analyzed by gas chromatography-mass spectrometry (GC-MS). The temperature for GC-MS analysis was initially held at 50° C. for 2 min, ramped to 200° C. at 25° C./min, held for 1 min, then raised to 315° C. at 25° C./min, held for 2 min. Helium was used as a carrier gas and the flow rate was 1 ml/min through a DB-5MS separation column (30 m, 0.25 mm ID, 0.25 μm, Agilent). Methods for calculating average membrane lipid length and lipid saturated: unsaturated ratio can be found in [14].

Membrane Lipid Content Measurement: Thirty milliliters of mid-log E. coli cells were centrifuged, washed by ddH2O and adjusted to OD550˜10. Then, 1.8 ml of cell suspension was centrifuged at 14,000×g for 5 min and the resulting cell pellets were resuspended in 1.4 ml methanol. As described in 2.4, the total membrane bound fatty acid was measured. Given that membrane-bound fatty acids account for 71% (w/w) of lipid mass [47], we use the following formula to calculate the membrane lipid content: total membrane lipid (mg/g DCW)=membrane fatty acids (mg)/0.71×g DCW. 2.6 Real-time quantitative PCR: Bacterial cultures were grown and collected by centrifugation at 10,000×g for 2 min. Total RNA was extracted by using the RNeasy Mini Kit (Qiagen), and the residual DNA was removed by TURBO DNA-Free™ Kit (Life Technology). Superscript III First-Strand Synthesis SuperMix (Invitrogen) was employed for the cDNA synthesis, then the cDNA was diluted 100-fold and used as template for quantitative real-time PCR (qRT-PCR) analysis with SYBR Green ER™ qPCR SuperMix (Invitrogen). The E. coli 16S rrsA gene was employed as the housekeeping gene for fadL mRNA abundance analysis. Sequences of fadL primers for qRT-PCR are CTGAAATGTGGGAAGTGTC/GAAGGTCCAGTTATCATCGT, Primers for rrsA are TGGCTCAGATTGAACGC/ATCCGATGGCAAGAGGC. The qRT-PCR was performed with the StepOnePlus™ Real-Time PCR System (Thermo Fisher Scientific). The PCR mixture was held at 95° C. for 10 min and then subjected to 40 cycles of incubation at 95° C. for 15 s, then 60° C. for 1 min.

Fermentation for Fatty Acid Production: Individual colonies were selected from Luria Broth (LB) plates with ampicillin and inoculated into 3 ml of LB liquid medium with ampicillin for 4 h. Then, 0.5 ml of culture was added to 20 ml LB with ampicillin at 30° C., 220 rpm overnight for seed culture preparation. Seed culture was collected, resuspended in MOPS 2.0% (wt/v) dextrose medium, and transferred into 50 ml MOPS 2.0% (wt/v) dextrose containing ampicillin and 1 mM of isopropyl-β-D-thiogalactopyranoside (IPTG) in 250 ml baffled flasks. The target initial cell density was OD550-0.1. Cultures were grown in 250 ml baffled flasks with initial pH 7.0 at 30° C., 220 rpm for 72 h. 2.8 Determination of Carboxylic Acid Titers: Carboxylic acid production was quantified by an Agilent 7890 gas chromatograph equipped with an Agilent 5975 mass spectroscope using flame ionization detector and mass spectrometer (GC-MS) after carboxylic acid extraction. Briefly, 100 μL of whole liquid media sample was taken and 10 μL of 1 μg/μL C7:0/C11:0/C17:0 was added as internal standards. Two ml of ethanol: sulfuric acid (98:2 v/v) mixture was added, mixed and incubated at 65° C. for 30 minutes. Then, 2 ml of 0.9% (wt/v) NaCl solution and 1 ml of hexane were added, vortexed and centrifuged at 2,000×g for 2 min. The top hexane layer was then analyzed by GC-MS, as described in 2.2. 2.9 Statistical Analysis: The two-tailed t-test method was employed to analyze the statistical significance of all data in this study and P value <0.05 is deemed statistically significant.

### Results

Effects of ompF or fadL Deletion on Tolerance and Production of Fatty Acids

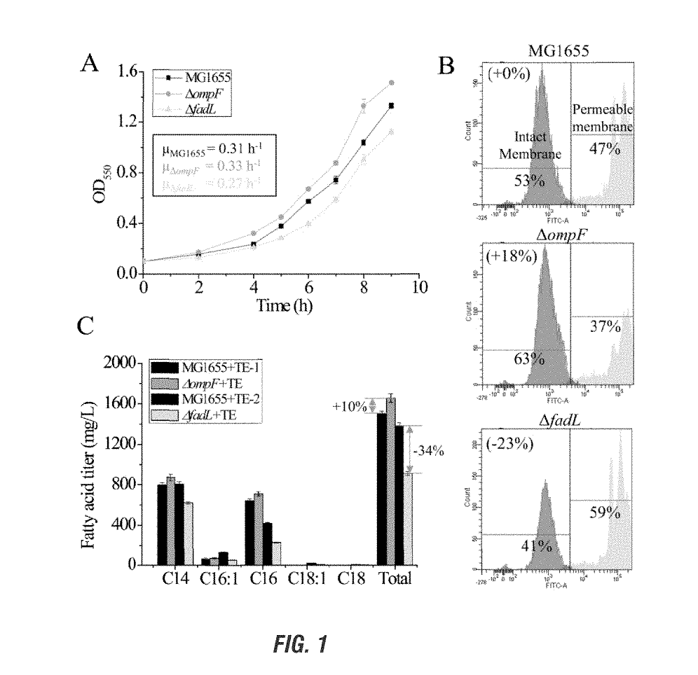

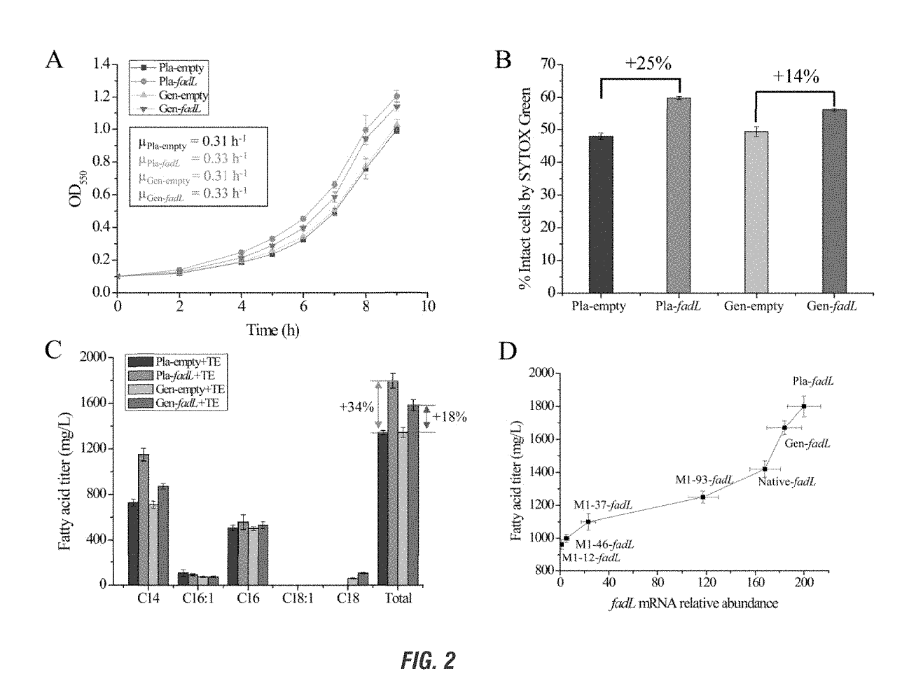

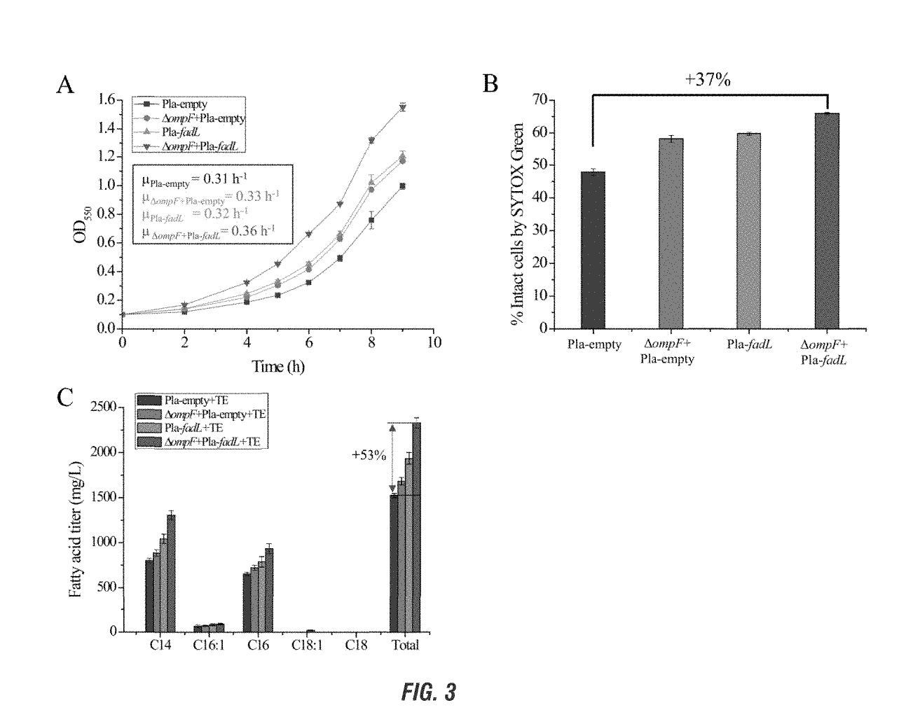

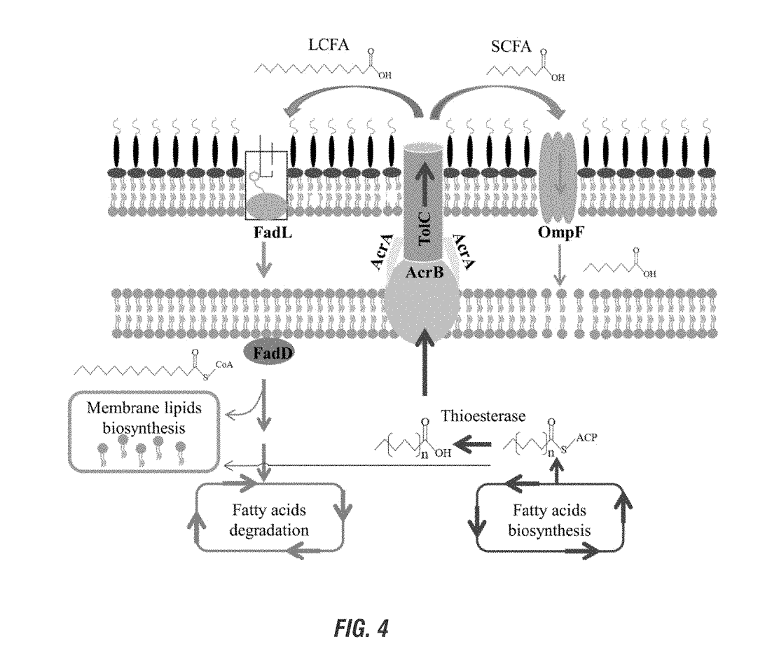

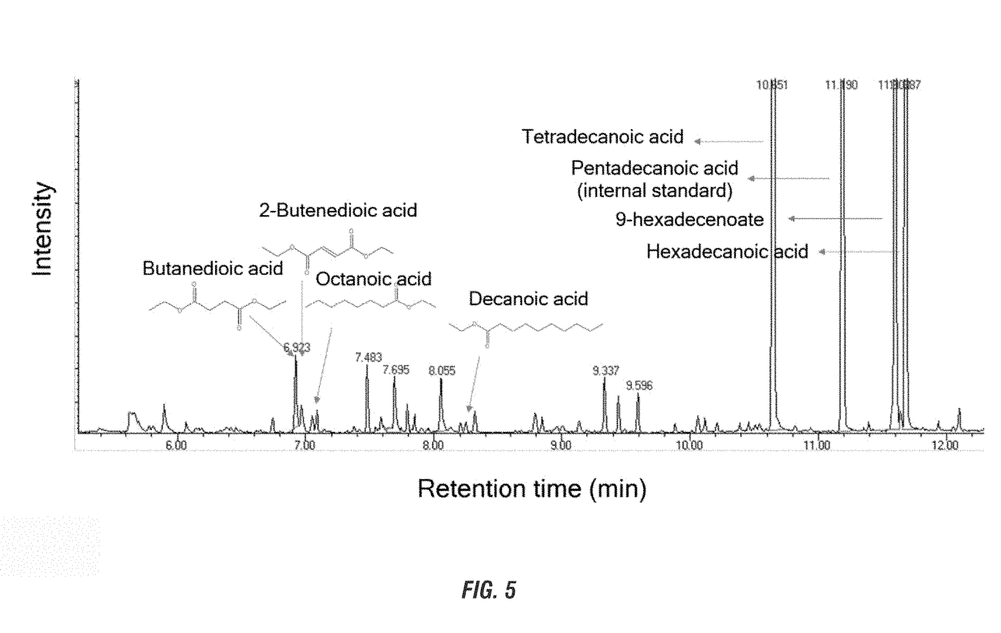

It was previously reported that OmpF facilitates transport of SCFA, such as octanoic acid (C8), into E. coli, and that deletion of ompF in E. coli BW25113 decreased the impact of C8 on biomass production [32]. To evaluate the effect of OmpF on C8 tolerance in MG1655, we also constructed an ompF (Genbank accession No. NC_000913.3) deletion strain (ΔompF) and confirmed that this engineering strategy improved tolerance to C8. In the absence of C8, the specific growth rates (μ) of both strains were approximately 0.39 h-1. During C8 challenge, the specific growth rate of the ΔompF mutant was 0.33 h-1, which is 7% higher than that of MG1655 (0.31 h-1) (FIG. 1A), which is consistent with the previous report [32].

Decreased membrane integrity has been previously described as a primary cause of C8 toxicity, where decreased membrane integrity is evidenced by leakage of metabolites and ions, such as Mg2+, out of the cell or the entry of membrane-impermeable molecules, such as SYTOX, into the cell [14, 24, 48]. We next characterized the membrane integrity changes after disruption of ompF. Consistent with the growth results, deletion of ompF dampened the impact of C8 on membrane integrity. Specifically, the percentage of cells with intact membranes, i.e. SYTOX impermeable, during challenge with exogenously provided 10 mM C8, increased by 18% compared with the wild-type control strain (P<0.05) (FIG. 1B).

Given that increased tolerance might lead to increased production of bio-products, we next applied the ompF deletion strategy to fatty acid production. The plasmid pXZ18Z (TE) harboring the heterologous thioesterase from Ricinus communis [42], which primarily releases tetradecanoic acid (C14:0), palmitoleic acid (C16:1) and hexadecanoic acid (C16:0), was transformed into the ΔompF strain and the corresponding control for fatty acid production in minimal MOPS 2.0% (wt/v) dextrose medium. We observed that deletion of ompF increased fatty acid production (FIG. 1C): in the ΔompF+TE mutant, the titer of C14:0 was increased by 10% (P=0.03) to 875 mg/L, C16:1 was increased by 17% (P=0.24) to 71 mg/L and C16:0 was increased by 11% (P=0.01) to 711 mg/L. All of these increases led to a 10% improvement of total fatty acids produced by the ΔompF+TE mutant compared to MG1655+TE strain, with titers of 1500±20 and 1660±40 mg/L, respectively (P=0.005). It should be noted that previous studies concluded that deletion of ompF from E. coli strain TY05 did not significantly increase fatty acid (C8-C14) production [26]. The difference from this previous report and the findings presented here may be due to the use of different thioesterases (from U. californica vs. from R. communis), growth media (nutrient-rich LB+0.4% (v/v) glycerol vs. minimal MOPS+2% (wt/v) glucose) and temperature (37 vs. 30° C.).

While OmpF has been previously characterized in terms of SCFA transport, FadL predominantly functions in the uptake of LCFA [29, 30]. To investigate the effect of FadL on fatty acid tolerance and production, a fadL deletion mutant (Fade) was constructed. Interestingly, the ΔfadL mutant showed decreased tolerance to C8. For example, the specific growth rate of the ΔfadL strain was 12% lower than that of MG1655 (0.27 vs. 0.31 h-1) (P<0.05) (FIG. 1A). Further membrane characterization showed that the percentage of cells with intact membranes was 23% lower for the ΔfadL strain than MG1655 (P<0.05) (FIG. 1B). When this fadL deletion strategy was applied to fatty acid production (+TE), titers of C14:0 decreased by 23% to 623 mg/L, C16:1 decreased by 60% to 51 mg/L and C16:0 decreased by 45% to 230 mg/L. Each of these changes had a P value less than 0.05. Together, these changes led to a 34% reduction of total fatty acids in the ΔfadL+TE mutant compared with MG1655+TE strain (from 1390±30 to 920±20 mg/L) (P<0.05) (FIG. 1C). It should be noted that the fatty acid titer of MG1655+TE here (1390±30 mg/L) is slightly lower than the 1500±20 mg/L described above for the ompF results, due to differences between batches, similar to the results described elsewhere [26]. As with deletion of ompF, our results differ from previous reports of the effect of fadL deletion on fatty acid production. This previous characterization employed E. coli strain TY05 in rich medium with glycerol and found no significant change in production of C8-C14 fatty acids upon deletion of fadL [26]. However, our observation that deletion of fadL can increase sensitivity to membrane-damaging short-chain fatty acids is consistent with observations made for phenylpropanoid tolerance [33].

Increased Expression of fadL Increased Fatty Acid Tolerance and Production

Given that the deletion of fadL decreased fatty acid tolerance and production, it is reasonable to expect that increased expression of fadL might improve fatty acid tolerance and production. To this end, two different strategies were employed in E. coli MG1655 for increased expression of fadL: plasmid expression (Pla-fadL) and genomic integration of a second copy of fadL (Gen-fadL). Consistent with our hypothesis, both of these increased expression strategies significantly improved C8 tolerance. Specifically, the specific growth rate of Pla-fadL (0.33 h-1) and Gen-fadL (0.33 h-1) were 8% and 7% higher than Pla-empty (0.31 h-1) and Gen-empty (0.31 h-1) (P<0.05) (FIG. 2A). Membrane damage, as evidenced by entry of the SYTOX nucleic acid dye into the cell, was decreased in the two strains engineered for increased fadL expression. Specifically, Pla-fadL showed a 25% increase in membrane integrity and Gen-fadL had a 14% increase in membrane integrity (P<0.05) (FIG. 2B).

Further characterization showed that both of the strains with increased fadL expression also had increased fatty acid production capability. This significantly (P<0.05) increased fatty acid titer was observed for C14:0 and the total fatty acid pool, though the increase was slightly higher for C16:1 and C16:0 in both cases (FIG. 2C). Specifically, the plasmid-based strain produced 1150 mg/L of C14:0, 556 mg/L of C16:0 and 1800 mg/L of total fatty acid, which was 57%, 10% and 34% higher than the corresponding control encoding the thioesterase and an empty plasmid. This control strain produced 728 mg/L C14:0, 505 mg/L C16:0 and 1340 mg/L total fatty acids. A similar trend was also observed for genome-based fadL expression tuning: 872 mg/L of C14:0, 531 mg/L of C16:0 and 1580 mg/L of total fatty acids were produced by the engineered Gen-fadL+TE strain, which was 23%, 6% and 18% higher than in the 710, 500 and 1340 mg/L produced by the corresponding Gen-empty+TE control. These results demonstrate the effectiveness of increasing fadL expression for increasing fatty acid production.

In order to further characterize the relationship between the expression level of fadL and fatty acid production, additional strains were constructed (+TE) and characterized. Specifically, different promoters (M1-12, M1-37, M1-46, M-93) with varied strengths [6, 39, 40] were employed to regulate the expression of the native fadL (FIG. 2D). A positive relationship between mRNA relative abundance of fadL and fatty acid titers was observed (FIG. 2D). For instance, mRNA relative abundance of fadL increased nearly 120-fold in M1-93-fadL strain relative to M1-12-fadL (of which fadL expression level was deemed as 1), and it also produced 1250 mg/L of fatty acid, which is 37% higher than the 915 mg/L produced by M1-12-fadL. It should be noted that expression level of fadL under all artificial promoters used here is lower than the native promoter, which suggests that expression of fadL is held at a relatively high level in E. coli MG1655.

Deletion of ompF and Increased Expression of fadL have an Additive Effect in Increasing Fatty Acid Production

Given that deletion of ompF and increased expression of fadL were each found to increase tolerance and production of fatty acids, we proposed that combinatorial utilization of both engineering strategies would further increase performance. To this end, the plasmid-based expression of fadL was selected as the strategy for increasing expression of fadL, due to its substantial increase in tolerance and production of fatty acid. Consistent with our hypothesis, combinatorial utilization of the ompF deletion and increased expression of fadL was found to have an additive effect for improving tolerance to C8 (FIG. 3A). The specific growth rate of ΔompF+Pla-fadL strain reached up to 0.36 h-1 in the presence of 10 mM C8, which exceeds that of Pla-empty=0.31 h-1) by 18%, and is also 10% higher than individual deletion of ompF (ΔompF+Pla-empty, μ=0.33 h-1) and 12% higher than individual increased expression of fadL (Pla-fadL, μ=0.32 h-1) (P<0.05) (FIG. 3A). Besides increased tolerance, membrane integrity was significantly increased in the ΔompF+Pla-fadL strain during challenge with C8. Compared with Pla-empty, the percentage of ΔompF+Pla-fadL cells with intact membranes increased by 37% (P<0.05) (FIG. 3B).

Combination of ompF deletion and increased expression of fadL also increased the specific growth rate during fatty acid production (data not shown), and final fatty acid titers (FIG. 3C). Specifically, the combination of these engineering strategies in the ΔompF+Pla-fadL+TE strain resulted in a specific growth rate of 0.25 h-1 in the first 12 h of fermentation, where this exceeds that of Pla-empty=0.16 h-1) by 53% (P<0.05). Correspondingly, the ΔompF+Pla-fadL+TE strain produced 1,310 mg/L of C14:0, 90 mg/L of C16:1, 930 mg/L of C16:0 and 2330 mg/L of total fatty acids after 72 h fermentation. These titers are 47%, 25%, 29% and 38% higher than the strain in which only the ompF deletion was implemented (ΔompF+Pla-empty+TE, 885 mg/L of C14:0, 72 mg/L of C16:1, 722 mg/L of C16:0 and 1680 mg/L of total fatty acid) and 25%, 10%, 18% and 20% higher than the strain in which only the fadL overexpression was implemented (Pole-vaulter, 1040 mg/L of C14:0, 83 mg/L of C16:1, 786 mg/L of C16:0 and 1930 mg/L of total fatty acid). Note that all of these comparisons have P<0.05, except for C16:1. The combined strain has an approximately 50% improvement in fatty acid titers relative to the corresponding un-engineered control, Pla-empty+TE, which produced 801 mg/L of C14:0, 65 mg/L of C16:1, 653 mg/L of C16:0 and 1520 mg/L of total fatty acid (FIG. 3C). These results again demonstrate the effectiveness of concurrent utilization of ompF deletion and increased expression of fadL for increasing fatty acid production.

### Functional Mechanism of OmpF and FadL on Increased Membrane Integrity

In this study, engineering the abundance of the membrane proteins OmpF and FadL increased membrane integrity, fatty acid tolerance and fatty acid production. Prior studies showed that increasing the average length or the saturated: unsaturated (S/U) ratio of E. coli membrane lipids can alleviate the decreased membrane integrity caused by fatty acids [23, 24]. In order to determine whether the increased membrane integrity here could be attributed to such changes in the phospholipid tail distribution, we measured the membrane lipid composition in the wild-type MG1655, ΔompF, ΔfadL and Pla-fadL strains (Table 3).

TABLE 3
Membrane lipid content and composition changes in the wild type MG1655, ΔompF, ΔfadL, Pla-fadL strains

| | Membrane lipid | | Membrane lipid composition (mol %) | | | | | | | Membrane | Membrane lipid |
|---|---|---|---|---|---|---|---|---|---|---|---|
| Strain | content (mg/g DCW) | | C14:0 | C16:1 | C16:0 | C17cyc | C18:1 | C18:0 | C19cyc | lipid length | S/U ratio |
| MG1655 | 69.4 ± 0.3 | | 1.3 ± 0.1 | 13.6 ± 0.2 | 48.5 ± 0.2 | 14.1 ± 0.1 | 19.1 ± 0.4 | 1.70 ± 0.03 | 1.8 ± 0.1 | 16.4 ± 0.2 | 1.06 ± 0.02 |
| ΔompF | 71.3 ± 0.5 | (+2.7%) | 1.1 ± 0.1 | 13.6 ± 0.1 | 48.7 ± 0.1 | 13.6 ± 0.3 | 19.4 ± 0.1 | 1.9 ± 0.1 | 1.7 ± 0.1 | 16.4 ± 0.1 | 1.07 ± 0.01 |
| ΔfadL | 62 ± 3 | (−10%) | 1.2 ± 0.1 | 12.7 ± 0.1 | 48.3 ± 0.4 | 14.5 ± 0.1 | 19.4 ± 0.3 | 1.9 ± 0.1 | 1.9 ± 0.1 | 16.4 ± 0.2 | 1.06 ± 0.01 |
| Pla-fadL | 78 ± 1 | (+13%) | 1.3 ± 0.1 | 13.2 ± 0.2 | 48.2 ± 0.1 | 13.4 ± 0.1 | 20.7 ± 0.1 | 1.9 ± 0.2 | 1.2 ± 0.1 | 16.4 ± 0.1 | 1.00 ± 0.02 |

Each value is an average and standard deviation of three biological replicates
All experiments were performed in MOPS +2% (wt/v) dextrose shake flasks at 220 rpm 30° C. with an initial pH of 7.0, 10 mM octanoic acid (C8). All values
are the average of at least three biological replicates with the associated standard deviation indicated. Percent increase values are only shown for differences
that were deemed statistically significant (P < 0.05)
DCW dry cell weight, S/U ratio membrane saturated: unsaturated lipid ratio

However, no significant changes in membrane composition were observed. Similarly, the average lipid length in wild-type MG1655 was 16.4±0.2, which is comparable to the value observed for the ΔompF, ΔfadL and Pla-fadL strains (Table 3). Additionally, the membrane lipid S/U ratio in the wild-type MG1655 was 1.06±0.02, which is similar to the ratios for the ΔompF, ΔfadL and Pla-fadL strains (Table 3). These results indicate that the previously-described membrane engineering mechanisms of increasing the membrane lipid and S/U ratio are not the underlying reason for increased membrane integrity here.

Since the membrane consists of lipids and proteins, altering the abundance of FadL and OmpF might affect the total membrane lipid content. The ΔompF strain had a comparable membrane lipid content to MG1655 (Table 3), which indicates that ompF deletion did not significantly impact membrane lipid production. However, unlike ompF, altering the abundance of fadL remarkably affected membrane lipid content. For example, the membrane lipid content of ΔfadL is only 62±3 mg/g DCW, which is an 11% decrease compared to MG1655 (P<0.05). Consistently, Pla-fadL had a 13% increase in membrane lipid content relative to MG1655 (P<0.05) (Table 3). This result indicates that, unlike OmpF, FadL might be involved in membrane lipid synthesis, and therefore altering the abundance of fadL affects the membrane lipid content and thus membrane integrity. It should be noted that the relative distribution of the lipid tails is not changed in the Pla-fadL strain (Table 3).

### DISCUSSION

Product toxicity is often an obstacle for cost-effective production of biofuels and chemicals [9, 10]. Therefore, construction of robust production organisms tolerant to these biorenewables is critical for industrial applications and has attracted increasing attention in recent years [12, 45, 49, 50]. Given its importance to overall cell function, membrane integrity has become an attractive engineering target for enhancing robustness [13, 24]. In the case of fatty acids, a variety of engineering efforts have been applied to increasing membrane integrity, with mixed results. Most of these engineering strategies focused on altering the distribution of the membrane lipids of E. coli, such as by altering the average lipids length or degree of saturation [23, 24], though there have also been efforts to identify an efflux system that can improve fatty acid production [26].

Here we focused on two membrane proteins, OmpF and FadL, and found that they have distinct effects on maintaining membrane integrity during fatty acid challenge and production. OmpF has been reported to function as the general diffusion porin of E. coli, through which a variety of inhibitory molecules, e.g. antibiotics, colicin and SCFA, can enter the cell [32, 51, 52]. Rodriguez-Moya et al. showed that OmpF facilitates transport of C8 into E. coli, disrupting intracellular pH and oxidative balance [32]. It has also been suggested that OmpF is involved in the removal of phenylpropanoids from the cell interior [33]. In this study, we further characterized the role of OmpF in maintaining membrane integrity and used the ompF deletion strategy to increase fatty acid production. Although we employed the thioesterase specific for release of LCFA (C14-C16), some SCFAs were produced (e.g. C8 and C10) (FIG. 5). These endogenously produced SCFAs can be exported, i.e. by AcrAB-TolC [26], to the extracellular environment. Conversely, they can also re-enter across the outer membrane through E. coli porins (e.g. OmpF) (FIG. 4), which can cause severe membrane damage to E. coli even at low concentrations [14].

One possible explanation for our observations is that after the endogenously produced fatty acids exit the cell, presumably via ArcAB-TolC [26], some of the SCFA re-enter the cell via OmpF. Deletion of ompF blocks this re-entry and thereby increases membrane integrity, which in turn reduces the leakage of important cellular molecules such as Mg2+[14, 53], thereby elevating fatty acid tolerance and production (FIG. 4). The unexpected driving force for such transport may be due to the nature of the AcrAB-TolC transporter. Specifically, this transporter spans the periplasmic space [54-56] and thus the periplasm should be relatively depleted in fatty acids.

Our results demonstrate that, in addition to membrane engineering strategies that alter the distribution of the membrane lipid tails, altering the abundance of membrane protein OmpF can also affect membrane integrity and production of fatty acids, which provides another strategy for future membrane engineering. Increasing the expression of an efflux pump has been shown to improve the production of inhibitory products, such as valine [25] and limonene [8] and these efflux pumps are also an important part of antibiotic resistance [57]. To the best of our knowledge, this is the first demonstration that deletion of a transporter is associated with increased production of a membrane-damaging compound. In contrast to the ompF deletion strategy, deletion of fadL was found to decrease membrane integrity, tolerance and production of fatty acid. FadL is the only known outer membrane protein capable of importing exogenous hydrophobic LCFA compounds in E. coli [32, 34, 58, 59]. Imported LCFA can be degraded through the β-oxidation pathway as sources of carbon and energy, or serve as precursors for membrane phospholipid biosynthesis [30, 59-61]. Since there was still residual glucose at the end of our experiments (data not shown), it is not likely that the decreased fatty acid tolerance and decreased fatty acid production of the ΔfadL mutant was caused by carbon or energy limitations. Membrane lipid biosynthesis in E. coli requires acyl chains (C16:0, C16:1 and C18:1), of which there are two sources: (1) endogenous long chain acyl-ACP produced by the fatty acid biosynthesis pathway; and (2) long chain acyl-CoA derived from exogenous LCFA [62, 63]. Upon inactivation of FadL, uptake of exogenous LCFA will be decreased and thus membrane lipid biosynthesis will be impaired (FIG. 4). Our experimental results verify this hypothesis, as membrane lipid content was decreased in the ΔfadL strain and increased in the Pla-fadL strain. Since lipids are the primary structural component of the membrane, changing the membrane lipid content is likely to alter the membrane integrity. This altered membrane lipid content by ΔfadL or Pla-fadL does not change the distribution of the different membrane lipid types (Table 3), which suggests that FadL is only responsible for supplying LCFA precursors instead of directly participating in the biosynthesis of phospholipids.

As with OmpF, a driving force for fatty acid uptake via FadL is not expected to exist during fatty acid production. Here, we again refer to the nature of the AcrAB-TolC efflux pump as a possible reason for the existence of this driving force. Since the AcrAB-TolC system spans the periplasmic space [54-56], the periplasm may be depleted of fatty acids relative to the extracellular medium. This direct relationship between fadL expression and tolerance of membrane-damaging compounds has been noted elsewhere, specifically in regard to phenylpropanoids [33]. This protective effect of FadL against rutin, naringenin and resveratrol was attributed to FadL's role in repairing membrane damage, though there is no apparent exogenous source of the fatty acids used for this membrane repair [33]. Current membrane engineering strategies focus on altering membrane lipids composition, such as with the goal of increasing membrane lipid length or S/U ratio, to increase membrane integrity. Our results show that increasing the whole membrane lipid content possibly also contributes to increased membrane integrity, tolerance and production of fatty acids, which may serve as a novel strategy for membrane engineering in the future. Our qRT-PCR results showed that there is a positive relationship between fadL mRNA abundance and fatty acid titer, and they also show that the native fadL gene is maintained at a high expression level, which indicates the importance of FadL in maintaining normal phospholipids biosynthesis. Concurrent deletion of ompF and increased expression of fadL synergistically increased fatty acid tolerance and production, accompanied by increased membrane integrity, possibly due to an increase in membrane lipid content and prevention of re-entry of the SCFA.

Bae et al. found that deletion of fadD and overexpression of fadL in E. coli increased hydroxy long-chain fatty acid production [34]. In that study, it was concluded that overexpression of fadL contributes to the improvement in the production of ω-hydroxy palmitic acid, primarily due to increased ability to transport exogenously fed palmitic acid (C16). The present work mainly focuses on the effect of fadL overexpression on the import of exogenous LCFA for membrane lipid synthesis and thus maintaining membrane integrity during the production of or challenge with membrane-damaging fatty acids. Prior research showed that deletion of ompF or fadL in E. coli did not affect fatty acid production [26], which is different from our results. There are two possible reasons for this difference: (A) the use of different thioesterases; and (B) the use of different growth conditions. The previous studies used a C8-C14-producing thioesterase enzyme from Umbellularia californica, while here we used a C14-C16-producing thioesterase from Ricinus communis. This previous study also used nutrient-rich LB with 0.4% (v/v) glycerol at 37° C., while we used the nutrient-poor minimal MOPS with 2% (wt/v) dextrose at 30° C. It is interesting to note that the studies that identified a positive relationship between OmpF abundance, FadL abundance and phenylpropanoid tolerance were also performed at 30° C. [33]. The use of glycerol in the previous fatty acid production studies may also be a complicating factor. The increase in hydroxy-palmitic acid production upon overexpression of FadL was smaller in the presence of glycerol relative to glucose [34] and the presence of glycerol has previously been reported to alter the phospholipid composition of microbial cell membranes [64-66]. Under different growth conditions, the membrane composition and associated amount of membrane damage caused by the fatty acids is expected to vary, and therefore the roles of OmpF and FadL may differ.

This engineering method appears to increase fatty acid production as a direct function of increased abundance of the microbial biocatalyst. Thus, it differs from a previously described membrane engineering method that increased fatty acid titers by 50% without impacting the final culture OD [23] and evolutionary strain development that improved fatty acid production 5-fold while only increasing growth during fatty acid production 3-fold [50]. The strategy described here also differs from provision of valine-producing E. coli with a valine exporter, which increased valine titers by 50% without changing the final OD [25]. Thus, additional strain engineering would be needed in order for this strategy to be effective in improving fatty acid production in fed-batch or continuous culture systems. However, this work clearly demonstrates that these two membrane proteins are two viable engineering targets for improving fatty acid production.

### CONCLUSIONS

Membrane damage of the microbial biocatalyst is a widespread problem in the problem of biorenewable fuels and chemicals. Here we have demonstrated two strategies for dealing with membrane damage in our condition. The first is to increase the abundance of FadL, which we propose increases the ability of the organism to repair the membrane damage incurred by fatty acids. The second method is to delete OmpF, which we propose prevents re-entry of the inhibitory product.

### List of Abbreviations

OmpF, outer membrane porin F; FadL, long-chain fatty acid outer membrane porin; SCFA, short-chain fatty acids; LCFA: long chain fatty acids; C7, heptanoic acid; C8, octanoic acid; C11, undecanoic acid; C14, tetradecanoic acid; C15, pentadecanoic acid; C16:1, palmitoleic acid; C16:0, hexadecanoic acid; C17, heptadecanoic acid; TE, pXZ18Z; DCW, dry cell weight; IPTG, isopropyl-β-D-thiogalactopyranoside; ORF, open reading frame; ldhA, lactate dehydrogenase gene.

### REFERENCES

1. Gallezot P: Process options for converting renewable feedstocks to bioproducts. Green Chemistry 2007, 9:295-302.
2. Larson E D: A review of life-cycle analysis studies on liquid biofuel systems for the transport sector. Energy for Sustainable Development 2006, 10:109-126.
3. Thakker C, Martinez I, San K Y, Bennett G N: Succinate production in Escherichia coli. Biotechnology Journal 2012, 7:213-224.
4. Park J, Rodriguez-Moya M, Li M, Pichersky E, San K Y, Gonzalez R: Synthesis of methyl ketones by metabolically engineered Escherichia coli. J Ind Microbiol Biotechnol 2012, 39:1703-1712.
5. McKenna R, Nielsen D R: Styrene biosynthesis from glucose by engineered E. coli. Metab Eng 2011, 13:544-554.
6. Zhu X, Tan Z, Xu H, Chen J, Tang J, Zhang X: Metabolic evolution of two reducing equivalent-conserving pathways for high-yield succinate production in Escherichia coli. Metab Eng 2014, 24C:87-96.
7. Atsumi S, Cann A F, Connor M R, Shen C R, Smith K M, Brynildsen M P, Chou K J, Hanai T, Liao J C: Metabolic engineering of Escherichia coli for 1-butanol production. Metab Eng 2008, 10:305-311.
8. Dunlop M J, Dossani Z Y, Szmidt H L, Chu H C, Lee T S, Keasling J D, Hadi M Z, Mukhopadhyay A: Engineering microbial biofuel tolerance and export using efflux pumps.

Molecular Systems Biology 2011, 7.
9. Jarboe L R, Liu P, Royce L A: Engineering inhibitor tolerance for the production of biorenewable fuels and chemicals. Current Opinion in Chemical Engineering 2011, 1:38-42.
10. Nicolaou S A, Jaida S M, Papoutsakis E T: A comparative view of metabolite and substrate stress and tolerance in microbial bioprocessing: From biofuels and chemicals, to biocatalysis and bioremediation. Metab Eng 2010, 12:307-331.
11. Huffer S, Clark M E, Ning J C, Blanch H W, Clark D S: Role of Alcohols in Growth, Lipid Composition, and Membrane Fluidity of Yeasts, Bacteria, and Archaea. Applied and Environmental Microbiology 2011, 77:6400-6408.
12. Lennen R M, Kruziki M A, Kumar K, Zinkel R A, Burnum K E, Lipton M S, Hoover S W, Ranatunga D R, Wittkopp T M, Marner W D, 2nd, Pfleger B F: Membrane stresses induced by overproduction of free fatty acids in Escherichia coli. Appl Environ Microbiol 2011, 77:8114-8128.
13. Liu P, Chernyshov A, Najdi T, Fu Y, Dickerson J, Sandmeyer S, Jarboe L: Membrane stress caused by octanoic acid in Saccharomyces cerevisiae. Applied Microbiology and Biotechnology 2013, 97:3239-3251.
14. Royce L A, Liu P, Stebbins M J, Hanson B C, Jarboe L R: The damaging effects of short chain fatty acids on Escherichia coli membranes. Appl Microbiol Biotechnol 2013, 97:8317-8327.
15. Zaldivar J, Ingram L O: Effect of organic acids on the growth and fermentation of ethanologenic Escherichia coli LY01. Biotechnol Bioeng 1999, 66:203-210.
16. Cameron D C, Tong I T: Cellular and Metabolic Engineering—An Overview. Applied Biochemistry and Biotechnology 1993, 38:105-140.
17. Bailey J E: Toward a Science of Metabolic Engineering. Science 1991, 252:1668-1675.
18. Korstanje T J, van der Vlugt J I, Elsevier C J, de Bruin B: Hydrogenation of carboxylic acids with a homogeneous cobalt catalyst. Science 2015, 350:298-302.
19. Lennen R M, Braden D J, West R M, Dumesic J A, Pfleger B F: A Process for Microbial Hydrocarbon Synthesis: Overproduction of Fatty Acids in Escherichia coli and Catalytic Conversion to Alkanes. Biotechnology and Bioengineering 2010, 106:193-202.
20. Lopez-Ruiz J A, Davis R J: Decarbonylation of heptanoic acid over carbon-supported platinum nanoparticles. Green Chemistry 2014, 16:683-694.
21. Kim S, Cheong S, Chou A, Gonzalez R: Engineered fatty acid catabolism for fuel and chemical production. Current Opinion in Biotechnology 2016, 42:206-215.
22. Sanchez M A, Torres G C, Mazzieri V A, Pieck C L: Selective hydrogenation of fatty acids and methyl esters of fatty acids to obtain fatty alcohols-a review. Journal of Chemical Technology and Biotechnology 2017, 92:27-42.
23. Sherkhanov S, Korman T P, Bowie J U: Improving the tolerance of Escherichia coli to medium-chain fatty acid production. Metab Eng 2014, 25:1-7.
24. Lennen R M, Pfleger B F: Modulating Membrane Composition Alters Free Fatty Acid Tolerance in Escherichia coli. Plos One 2013, 8.
25. Park R I, Lee K H, Kim T Y, Lee S Y: Metabolic engineering of Escherichia coli for the production of L-valine based on transcriptome analysis and in silico gene knockout simulation. Proc Natl Acad Sci USA 2007, 104:7797-7802.
26. Lennen R M, Politz M G, Kruziki M A, Pfleger B F: Identification of transport proteins involved in free fatty acid efflux in Escherichia coli. J Bacteriol 2013, 195:135-144.
27. Cowan S W, Schirmer T, Rummel G, Steiert M, Ghosh R, Pauptit R A, Jansonius J N, Rosenbusch J P: Crystal structures explain functional properties of two E. coli porins. Nature 1992, 358:727-733.
28. Nikaido H: Outer-Membrane Barrier as a Mechanism of Antimicrobial Resistance. Antimicrobial Agents and Chemotherapy 1989, 33:1831-1836.
29. Lepore B W, Indic M, Pham H, Hearn E M, Patel D R, van den Berg B: Ligand-gated diffusion across the bacterial outer membrane. Proceedings of the National Academy of Sciences of the United States of America 2011, 108:10121-10126.
30. van den Berg B, Black P N, Clemons W M, Jr., Rapoport T A: Crystal structure of the long-chain fatty acid transporter FadL. Science 2004, 304:1506-1509.
31. Black P N: Characterization of FadL-Specific Fatty-Acid Binding in Escherichia coli. Biochimica Et Biophysica Acta 1990, 1046:97-105.
32. Rodriguez-Moya M, Gonzalez R: Proteomic analysis of the response of Escherichia coli to short-chain fatty acids. J Proteomics 2015, 122:86-99.
33. Zhou J W, Wang K, Xu S, Wu J J, Liu P R, Du G C, Li J H, Chen J: Identification of membrane proteins associated with phenylpropanoid tolerance and transport in Escherichia coli BL21. Journal of Proteomics 2015, 113:15-28.
34. Bae J H, Park B G, Jung E, Lee P G, Kim B G: fadD deletion and fadL overexpression in Escherichia coli increase hydroxy long-chain fatty acid productivity. Appl Microbiol Biotechnol 2014, 98:8917-8925.
35. Call T P, Akhtar M K, Baganz F, Grant C: Modulating the import of medium-chain alkanes in E. coli through tuned expression of FadL. Journal of Biological Engineering 2016, 10.
36. Heipieper H J, Keweloh H, Rehm H J: Influence of Phenols on Growth and Membrane-Permeability of Free and Immobilized Escherichia coli. Applied and Environmental Microbiology 1991, 57:1213-1217.
37. Zhang D F, Li H, Lin X M, Wang S Y, Peng X X: Characterization of Outer Membrane Proteins of Escherichia coli in Response to Phenol Stress. Current Microbiology 2011, 62:777-783.
38. Datsenko K A, Wanner B L: One-step inactivation of chromosomal genes in Escherichia coli K-12 using PCR products. Proceedings of the National Academy of Sciences of the United States of America 2000, 97:6640-6645.
39. Tang J, Zhu X, Lu J, Liu P, Xu H, Tan Z, Zhang X: Recruiting alternative glucose utilization pathways for improving succinate production. Appl Microbiol Biotechnol 2013, 97:2513-2520.
40. Tan Z, Zhu X, Chen J, Li Q, Zhang X: Activating phosphoenolpyruvate carboxylase and phosphoenolpyruvate carboxykinase in combination for improvement of succinate production. Appl Environ Microbiol 2013, 79:4838-4844.
41. Zhang X, Jantama K, Moore J C, Shanmugam K T, Ingram L O: Production of L-alanine by metabolically engineered Escherichia coli. Applied Microbiology and Biotechnology 2007, 77:355-366.
42. San K-Y, Li M, Zhang X: Bacteria and method for synthesizing fatty acids. Google Patents; 2011.
43. Neidhard. Fc, Bloch P L, Smith D F: Culture Medium for Enterobacteria. Journal of Bacteriology 1974, 119:736-747.
44. Wanner B L: Methods in Molecular Genetics. Academic; 1994.
45. Tan Z, Yoon J M, Nielsen D R, Shanks J V, Jarboe L R: Membrane Engineering via Trans Unsaturated Fatty Acids Production Improves Escherichia coli Robustness and Production of Biorenewables. Metab Eng 2016.
46. Bligh E G, Dyer W J: A Rapid Method of Total Lipid Extraction and Purification. Canadian Journal of Biochemistry and Physiology 1959, 37:911-917.
47. Torella J P, Ford T J, Kim S N, Chen A M, Way J C, Silver P A: Tailored fatty acid synthesis via dynamic control of fatty acid elongation. Proc Natl Acad Sci USA 2013, 110:11290-11295.
48. Lian J, McKenna R, Rover M R, Nielsen D R, Wen Z, Jarboe L R: Production of biorenewable styrene: utilization of biomass-derived sugars and insights into toxicity. J Ind Microbiol Biotechnol 2016, 43:595-604.
49. Chubukov V, Mingardon F, Schackwitz W, Baidoo E E K, Alonso-Gutierrez J, Hu Q J, Lee T S, Keasling J D, Mukhopadhyay A: Acute Limonene Toxicity in Escherichia coli Is Caused by Limonene Hydroperoxide and Alleviated by a Point Mutation in Alkyl Hydroperoxidase AhpC. Applied and Environmental Microbiology 2015, 81:4690-4696.
50. Royce L A, Yoon J M, Chen Y, Rickenbach E, Shanks J V, Jarboe L R: Evolution for exogenous octanoic acid tolerance improves carboxylic acid production and membrane integrity. Metab Eng 2015, 29:180-188.
51. Kim Y C, Tarr A W, Penfold C N: Colicin import into E. coli cells: A model system for insights into the import mechanisms of bacteriocins. Biochimica Et Biophysica Acta-Molecular Cell Research 2014, 1843:1717-1731.
52. Ziervogel B K, Roux B: The binding of antibiotics in OmpF porin. Structure 2013, 21:76-87.
53. Jarboe L R, Royce L A, Liu P: Understanding biocatalyst inhibition by carboxylic acids. Front Microbiol 2013, 4:272.
54. Du D J, Wang Z, James N R, Voss J E, Klimont E, Ohene-Agyei T, Venter H, Chiu W, Luisi B F: Structure of the AcrAB-TolC multidrug efflux pump. Nature 2014, 509:512-+.
55. Tikhonova E B, Zgurskaya H I: AcrA, AcrB, and TolC of Escherichia coli form a stable intermembrane multidrug efflux complex. Journal of Biological Chemistry 2004, 279:32116-32124.
56. Touze T, Eswaran J, Bokma E, Koronakis E, Hughes C, Koronakis V: Interactions underlying assembly of the Escherichia coli AcrAB-TolC multidrug efflux system. Molecular Microbiology 2004, 53:697-706.
57. Blair J M A, Richmond G E, Piddock L J V: Multidrug efflux pumps in Gram-negative bacteria and their role in antibiotic resistance. Future Microbiology 2014, 9:1165-1177.
58. Black P N, DiRusso C C: Transmembrane movement of exogenous long-chain fatty acids: proteins, enzymes, and vectorial esterification. Microbiol Mol Biol Rev 2003, 67:454-472, table of contents.
59. Hearn E M, Patel D R, Lepore B W, Indic M, van den Berg B: Transmembrane passage of hydrophobic compounds through a protein channel wall. Nature 2009, 458:367-U134.
60. Hearn E M, Patel D R, van den Berg B: Outer-membrane transport of aromatic hydrocarbons as a first step in biodegradation. Proceedings of the National Academy of Sciences of the United States of America 2008, 105:8601-8606.
61. Fujita Y, Matsuoka H, Hirooka K: Regulation of fatty acid metabolism in bacteria. Molecular Microbiology 2007, 66:829-839.
62. Rock C O: Fatty acid and phospholipid metabolism in prokaryotes. Biochemistry of Lipids, Lipoproteins and Membranes, 5th Edition 2008:59-96.
63. Rock C O, Jackowski S: Pathways for the Incorporation of Exogenous Fatty-Acids into Phosphatidylethanolamine in Escherichia coli. Journal of Biological Chemistry 1985, 260:2720-2724.
64. Du G C, Yang G, Qu Y B, Chen J, Lun S Y: Effects of glycerol on the production of poly (gamma-glutamic acid) by Bacillus licheniformis. Process Biochemistry 2005, 40:2143-2147.
65. Kautharapu K B, Rathmacher J, Jarboe L R: Growth condition optimization for docosahexaenoic acid (DHA) production by Moritella marina MP-1. Applied Microbiology and Biotechnology 2013, 97:2859-2866.
66. Pramanik J, Keasling J D: Effect of Escherichia coli biomass composition on central metabolic fluxes predicted by a stoichiometric model. Biotechnology and Bioengineering 1998, 60:230-238.

What is claimed is:

1. A genetically modified bacterium having improved membrane integrity and increased fatty acid production wherein said bacterium has increased FADL membrane content/activity and decreased OmpF membrane content/activity as compared to a non-modified bacterium.

2. The modified bacterium of claim 1 wherein said increase fatty acid production is primarily C14, C16:1 and C16 fatty acids.

3. The modified bacterium of claim 1 wherein said bacterium has decreased short chain fatty acid uptake and cellular content when compared to a non-modified bacterium

4. The bacterium of claim 1, comprising an expression construct with a FADL encoding nucleic acid sequence operably linked to a heterologous promoter.

5. The expression construct of claim 4 wherein said promoter is M1-12, MI-37, MI-46 or MI-93.

6. The expression construct of claim 4 further comprising a termination signal.

7. The expression construct of claim 4 further comprising an antibiotic resistance gene.

8. The bacterium of claim 1 wherein the OmpF gene is inactivated.

9. The bacterium of claim 1 wherein said OmpF gene includes a loss of function mutation.

10. The bacterium of claim 1 wherein said OmpF activity is decreased by an insertion, a deletion, of an amino acid in said protein or an interfering polypeptide.

11. The bacterium of claim 9 wherein said OmpF mutation is created by homologous recombination.

12. The bacterium of claim 11 wherein said OmpF gene is inactivated by CRISPRS or TALENS.

13. The bacterium of claim 9 wherein said mutation an insertion of deletion of one or more nucleotides in a gene encoding said protein.

14. The bacterium of claim 1, wherein said modified bacterium is created with host bacterium of MG1655.

15. The bacterium of claim 1, wherein the bacterium is selected from the group consisting of Gram negative bacteria and Gram-positive bacteria.

16. The bacterium of claim 15, wherein the Gram-negative bacterium is selected from the group consisting of Escherichia, Acinetobacter, Zymomonas, Gluconobacter, Geobacter, Shewanella, Salmonella, Enterobacter and Klebsiella.

17. The bacterium of claim 15, wherein the Gram-positive bacterium is selected from the group consisting of Bacillus, Clostridium, Corynebacterium, Lactobacillus, Lactococcus, Oenococcus, Streptococcus and Eubacterium.

18. The bacterium of claim 16, wherein the bacterium is Escherichia coli.

19. A method for producing long chain fatty acids comprising obtaining the bacteria of claim 1 and harvesting C14, C16:1, and/or C16 fatty acids from medium.

20. Long chain fatty acids produced by the method claim 19.

21. A method for altering fatty acids content as compared to wild-type, in a microbe, the method comprising transforming the microbe with a nucleotide construct comprising a polynucleotide construct for increasing FADL abundance/activity in an outer membrane, and a polynucleotide construct for decreasing OmpF abundance/activity in an outer membrane.

22. The method of claim 21, wherein the microbe is E. coli.

23. The method of claim 21, wherein the polynucleotide construct for increasing FADL abundance/activity further comprises an operably linked promoter that drives expression in a microbe cell.

24. The method of claim 21, wherein said polynucleotide sequence includes a FLP.

25. A transformed microbial cell produced by the process of claim 21.
